(19) United States
(12) Patent Application Publication
(10) Pub. No.: US 2024/0404075 A1
(43) Pub. Date: Dec. 5, 2024

(54) METHODS AND USER INTERFACES FOR CAMERA-BASED ASSESSMENT OF PHYSICAL MOVEMENT

(71) Applicant: Apple Inc., Cupertino, CA (US)

(72) Inventors: Marisa R. LU, San Francisco, CA (US); Matthew W. CROWLEY, Sunnyvale, CA (US); Mylene E. DREYER, Saint-Blaise (CH); Khristine V. GENDRON, Moraga, CA (US); Julian K. MISSIG, Burlingame, CA (US); Fiona P. O'LEARY, Palo Alto, CA (US); Jonathan A. TOPF, London (GB)

(21) Appl. No.: 18/615,954

(22) Filed: Mar. 25, 2024

Related U.S. Application Data
(60) Provisional application No. 63/470,444, filed on Jun. 1, 2023.

(51) Int. Cl. G06T 7/20; A63B 24/00; A63B 71/06; G06T 13/40; G06T 13/80; H04N 23/60

(52) U.S. Cl. CPC G06T 7/20; A63B 24/0062; A63B 71/0622; G06T 13/40; G06T 13/80; A63B 2071/0638; A63B 2220/05; A63B 2220/806; G06T 2207/30196; H04N 23/64

(57) ABSTRACT

The present disclosure generally relates to methods, systems, and user interfaces for camera placement and physical movement capture and methods, systems, and user interfaces for camera-based assessment of physical movement using a computer system.

### CROSS-REFERENCE TO RELATED APPLICATIONS

This application claims priority to U.S. Patent Application No. 63/470,444, entitled “METHODS AND USER INTERFACES FOR CAMERA-BASED ASSESSMENT OF PHYSICAL MOVEMENT,” filed on Jun. 1, 2023, the content of which are hereby incorporated by reference in their entirety.

### FIELD

The present disclosure relates generally to computer user interfaces, and more specifically to techniques for camera-based assessment of physical movement.

### BACKGROUND

Electronic devices can be used to assess the health and fitness of users. Such devices can use a variety of sensors to perform assessments, including cameras.

### BRIEF SUMMARY

Some techniques for camera-based assessment of physical movement using electronic devices, however, are generally cumbersome and inefficient. For example, some existing techniques use a complex and time-consuming user interface, which may include multiple key presses or keystrokes. Other techniques require a high degree of user skill or involvement to properly configure and/or position such devices to accurately assess physical movement or require the assistance of another person, often a trained professional. Moreover, some existing techniques require more time than necessary, wasting user time and device energy. This latter consideration is particularly important in battery-operated devices.

Accordingly, the present technique provides electronic devices with faster, more efficient methods and interfaces for camera-based assessment of physical movement. Such methods and interfaces optionally complement or replace other methods camera-based assessment of physical movement. Such methods and interfaces reduce the cognitive burden on a user and produce a more efficient human-machine interface. For battery-operated computing devices, such methods and interfaces conserve power and increase the time between battery charges.

In accordance with some embodiments, a method performed at a computer system that is in communication with a camera and an output device is described. The method includes: outputting, via the output device, guidance that directs a user of the computer system to place the camera at a target position within a physical environment; and after a determination is made that the camera meets a set of one or more placement criteria, capturing, via the camera, image data that includes physical movements of the user of the computer system.

In accordance with some embodiments, a non-transitory computer-readable storage medium storing one or more programs configured to be executed by one or more processors of a computer system that is in communication with a camera and an output device is described. The one or more programs including instructions for: outputting, via the output device, guidance that directs a user of the computer system to place the camera at a target position within a physical environment; and after a determination is made that the camera meets a set of one or more placement criteria, capturing, via the camera, image data that includes physical movements of the user of the computer system.

In accordance with some embodiments, a transitory computer-readable storage medium storing one or more programs configured to be executed by one or more processors of a computer system that is in communication with a camera and an output device is described. The one or more programs including instructions for: outputting, via the output device, guidance that directs a user of the computer system to place the camera at a target position within a physical environment; and after a determination is made that the camera meets a set of one or more placement criteria, capturing, via the camera, image data that includes physical movements of the user of the computer system.

In accordance with some embodiments, a computer system configured to communicate with a camera and an output device is described. The computer system comprising: one or more processors; and memory storing one or more programs configured to be executed by the one or more processors, the one or more programs including instructions for: outputting, via the output device, guidance that directs a user of the computer system to place the camera at a target position within a physical environment; and after a determination is made that the camera meets a set of one or more placement criteria, capturing, via the camera, image data that includes physical movements of the user of the computer system.

In accordance with some embodiments, a computer system configured to communicate with a camera and an output device is described. The computer system comprising: means for outputting, via the output device, guidance that directs a user of the computer system to place the camera at a target position within a physical environment; and means for, after a determination is made that the camera meets a set of one or more placement criteria, capturing, via the camera, image data that includes physical movements of the user of the computer system.

In accordance with some embodiments, a computer program product, comprising one or more programs configured to be executed by one or more processors of a computer system that is in communication with a camera and an output device is described. The one or more programs including instructions for: outputting, via the output device, guidance that directs a user of the computer system to place the camera at a target position within a physical environment; and after a determination is made that the camera meets a set of one or more placement criteria, capturing, via the camera, image data that includes physical movements of the user of the computer system.

In accordance with some embodiments, a method performed at a computer system that is in communication with a camera and a display generation component is described. The method comprising: capturing, via the camera, image data of a physical environment that includes a user of the computer system performing physical movements; assessing, based on the image data, whether the physical movements meet a set of one or more movement criteria; and after assessing whether the physical movements meet a set of one or more movement criteria, displaying, via the display generation component, visual feedback based on the image data.

In accordance with some embodiments, a non-transitory computer-readable storage medium storing one or more programs configured to be executed by one or more processors of a computer system that is in communication with a camera and a display generation component is described. The one or more programs including instructions for: capturing, via the camera, image data of a physical environment that includes a user of the computer system performing physical movements; assessing, based on the image data, whether the physical movements meet a set of one or more movement criteria; and after assessing whether the physical movements meet a set of one or more movement criteria, displaying, via the display generation component, visual feedback based on the image data.

In accordance with some embodiments, a transitory computer-readable storage medium storing one or more programs configured to be executed by one or more processors of a computer system that is in communication with a camera and a display generation component is described. The one or more programs including instructions for: capturing, via the camera, image data of a physical environment that includes a user of the computer system performing physical movements; assessing, based on the image data, whether the physical movements meet a set of one or more movement criteria; and after assessing whether the physical movements meet a set of one or more movement criteria, displaying, via the display generation component, visual feedback based on the image data.

In accordance with some embodiments, a computer system configured to communicate with a camera and a display generation component is described. The computer system comprising: one or more processors; and memory storing one or more programs configured to be executed by the one or more processors, the one or more programs including instructions for: capturing, via the camera, image data of a physical environment that includes a user of the computer system performing physical movements; assessing, based on the image data, whether the physical movements meet a set of one or more movement criteria; and after assessing whether the physical movements meet a set of one or more movement criteria, displaying, via the display generation component, visual feedback based on the image data.

In accordance with some embodiments, a computer system configured to communicate with a camera and a display generation component is described. The computer system comprising: means for capturing, via the camera, image data of a physical environment that includes a user of the computer system performing physical movements; means for assessing, based on the image data, whether the physical movements meet a set of one or more movement criteria; and means for, after assessing whether the physical movements meet a set of one or more movement criteria, displaying, via the display generation component, visual feedback based on the image data.

In accordance with some embodiments, a computer program product, comprising one or more programs configured to be executed by one or more processors of a computer system that is in communication with a camera and a display generation component is described. The one or more programs including instructions for: capturing, via the camera, image data of a physical environment that includes a user of the computer system performing physical movements; assessing, based on the image data, whether the physical movements meet a set of one or more movement criteria; and after assessing whether the physical movements meet a set of one or more movement criteria, displaying, via the display generation component, visual feedback based on the image data.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for camera-based assessment of physical movement, thereby increasing the effectiveness, efficiency, and user satisfaction with such devices. Such methods and interfaces may complement or replace other methods for camera-based assessment of physical movement.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3 is a block diagram of an exemplary multifunction device with a display and a touch-sensitive surface in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device, in accordance with some embodiments.

FIG. 6 is top-down schematic of a physical environment of a user, in accordance with some embodiments.

FIGS. 7A-7X illustrate exemplary user interfaces for camera placement and physical movement capture and also for camera-based assessment of physical movement using a computer system, in accordance with some embodiments.

FIG. 8 is a flow diagram of methods for camera placement and physical movement capture, in accordance with some embodiments.

FIG. 9 is a flow diagram of methods for camera-based assessment of physical movement using a computer system, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for camera-based assessment of physical movement. Such techniques can reduce the cognitive burden on a user who performs camera-based assessment of physical movement, thereby enhancing productivity. Further, such techniques can reduce processor and battery power otherwise wasted on redundant user inputs.

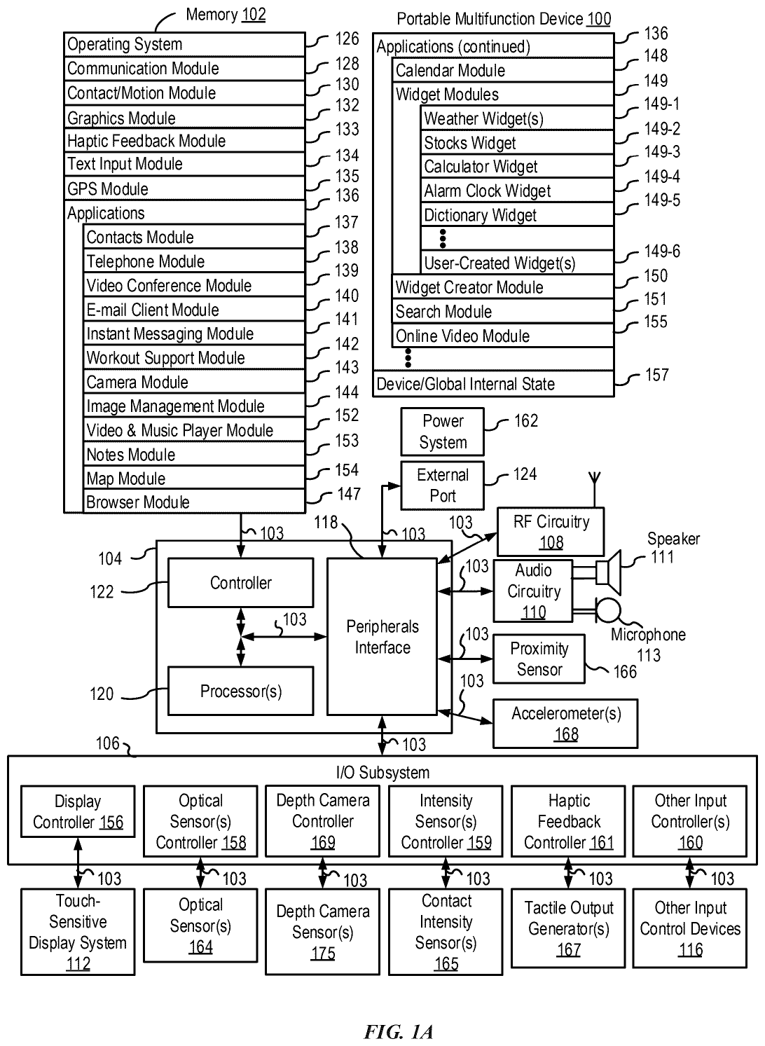

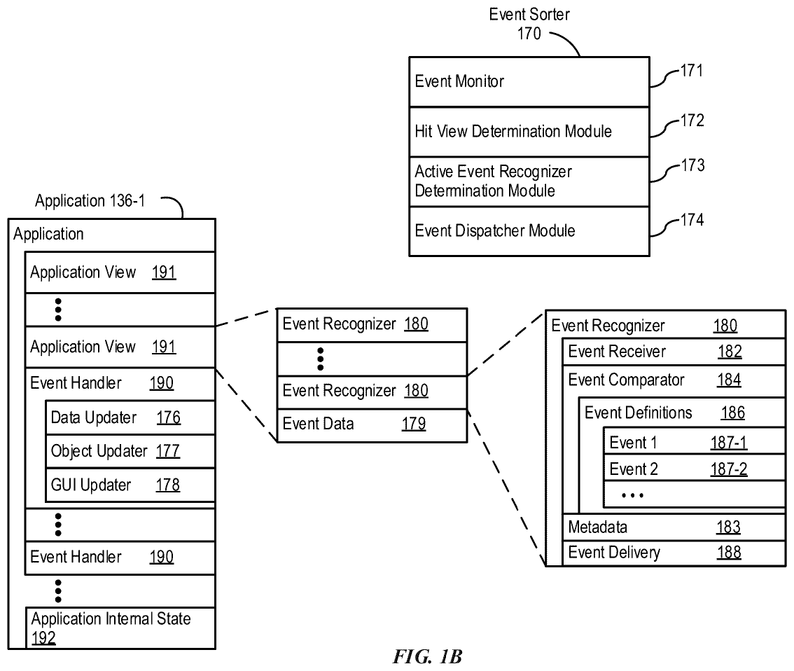

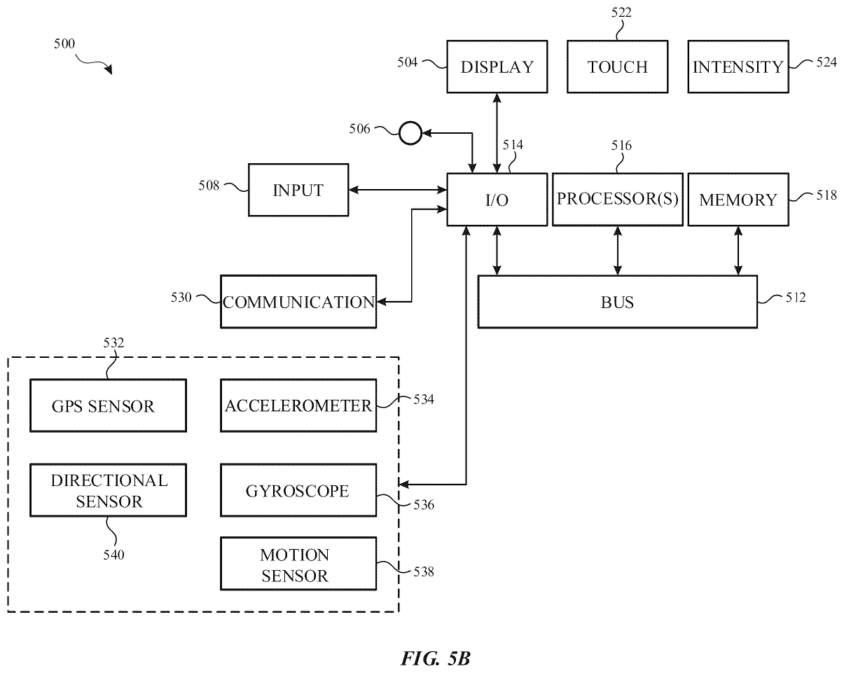

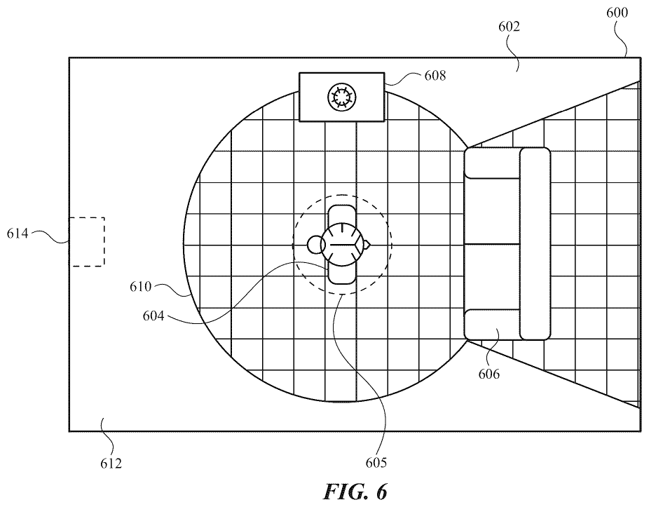

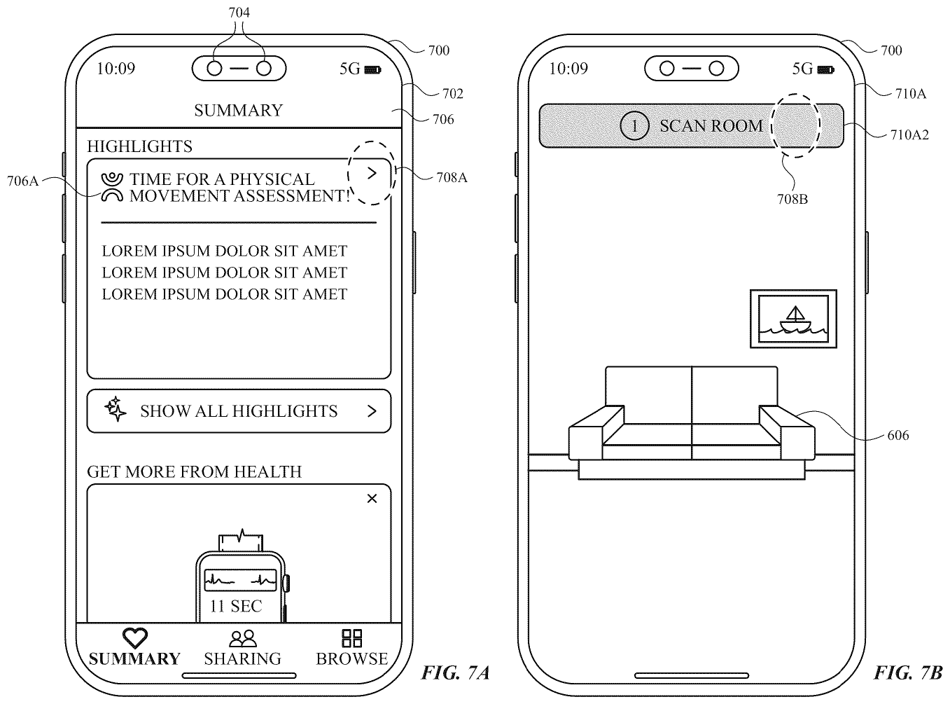

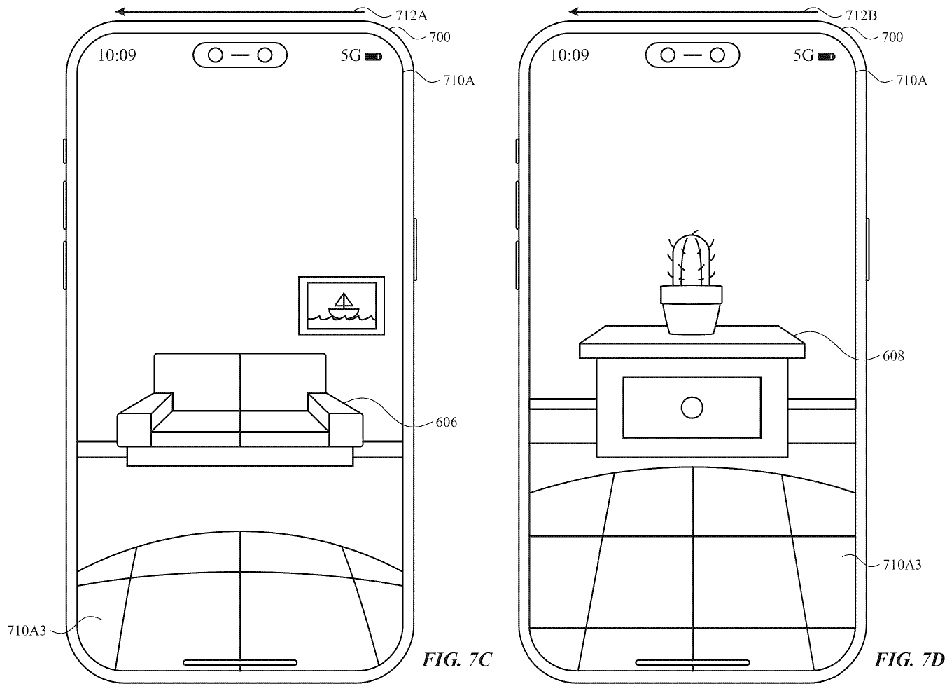

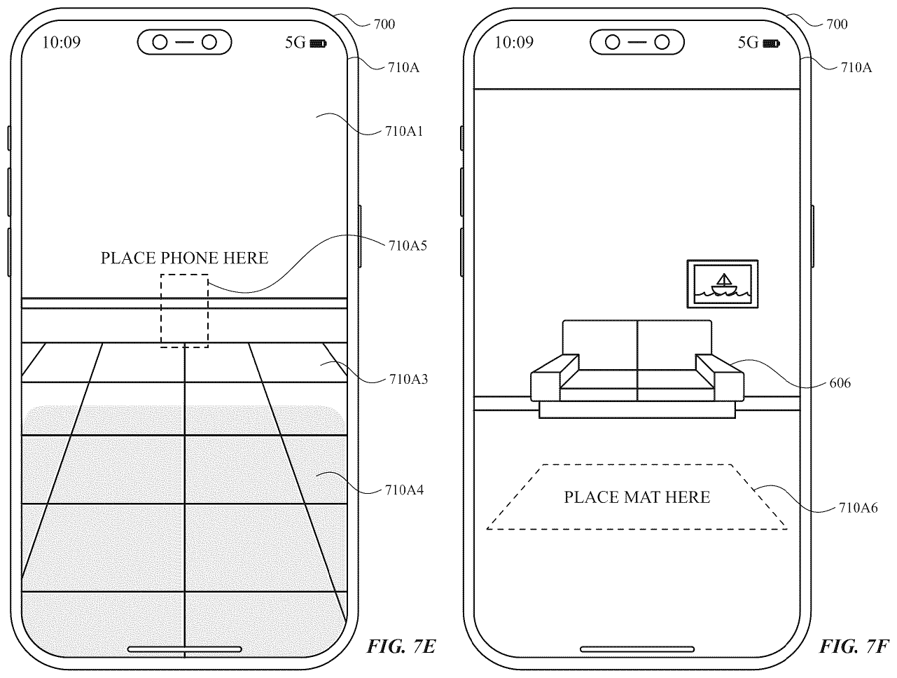

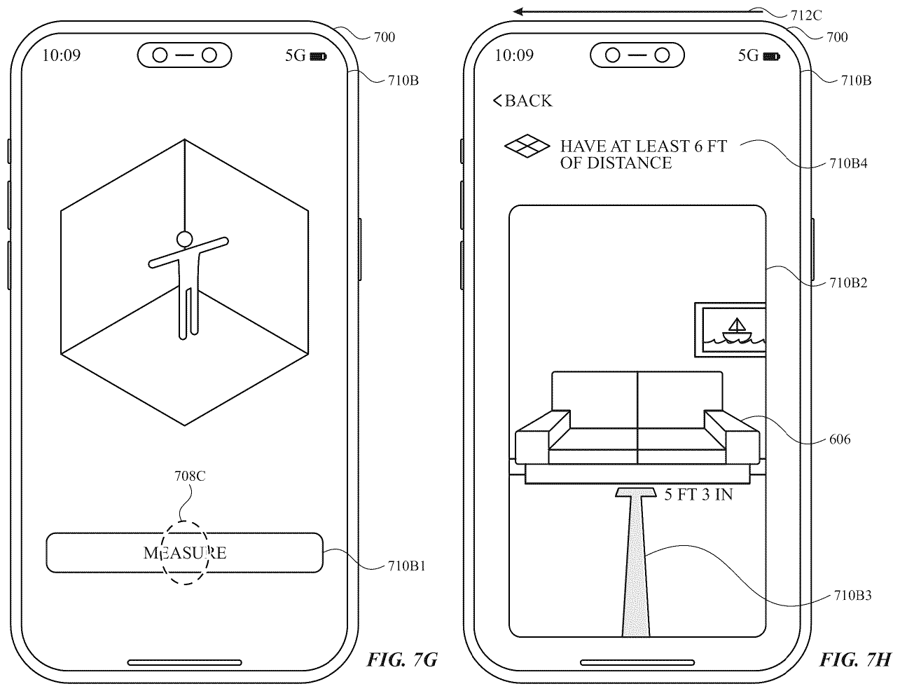

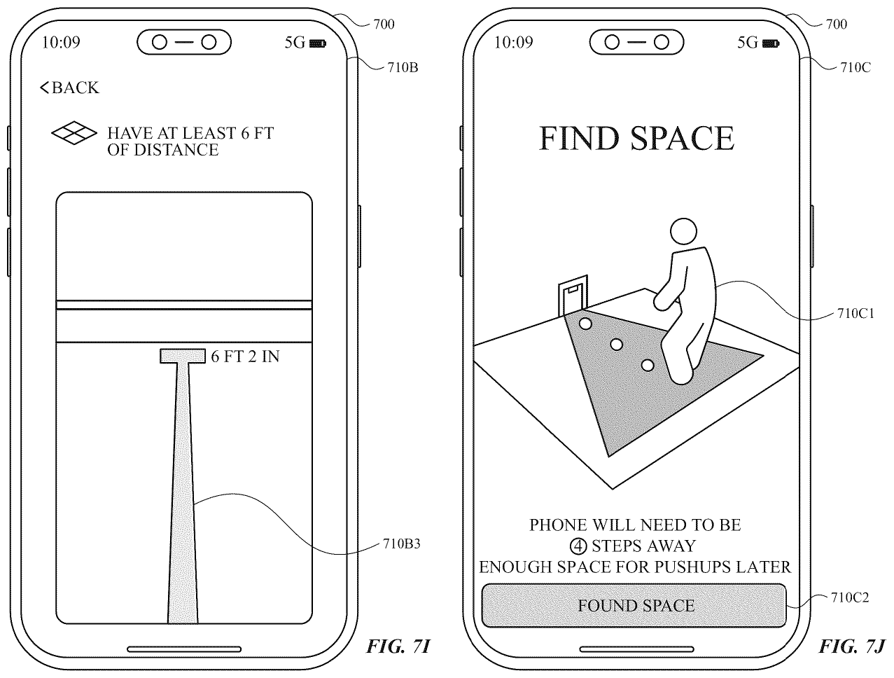

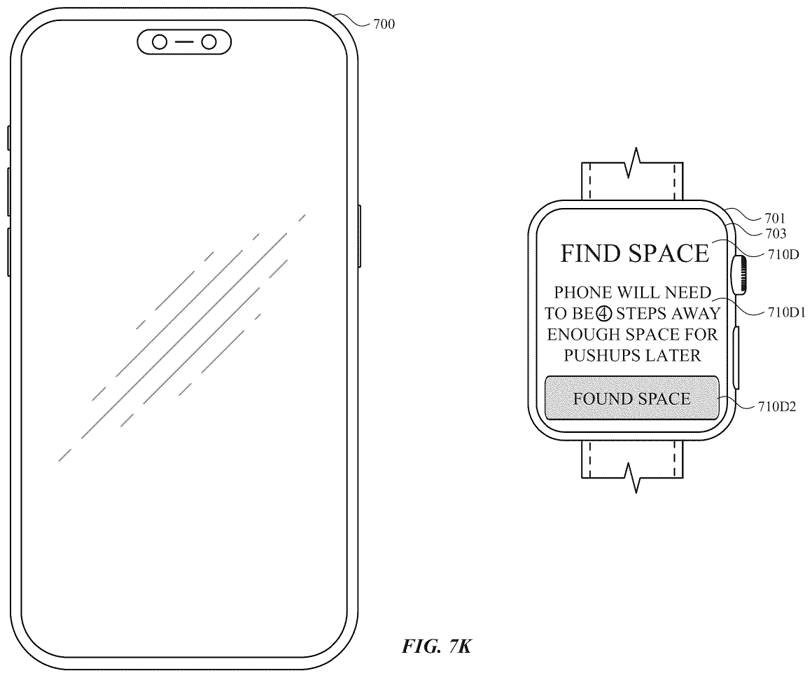

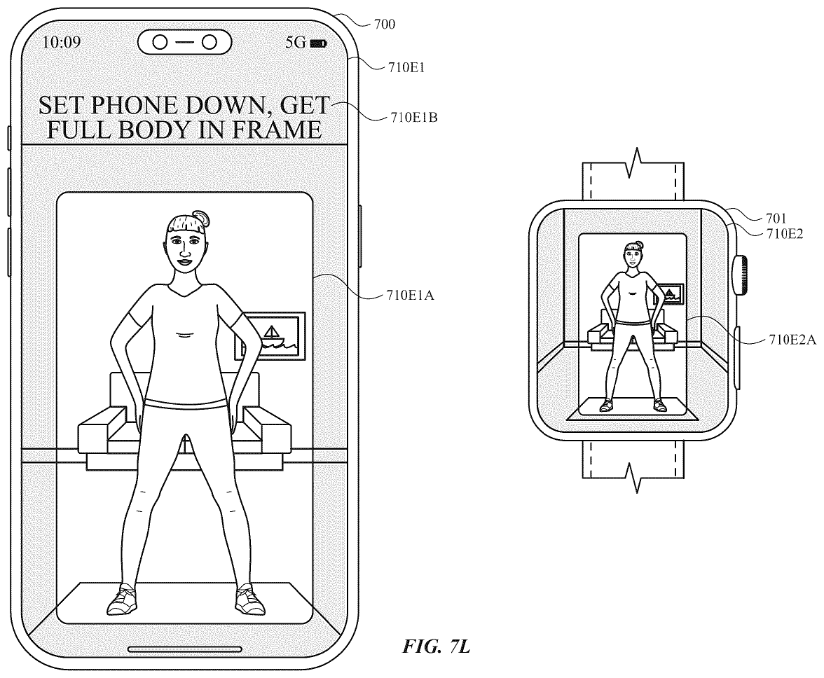

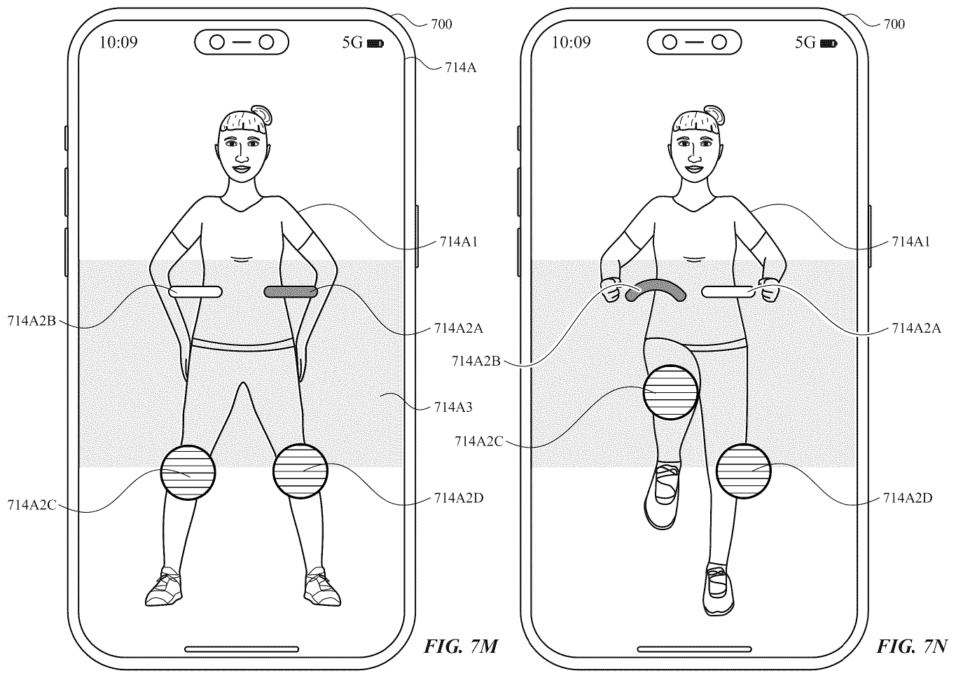

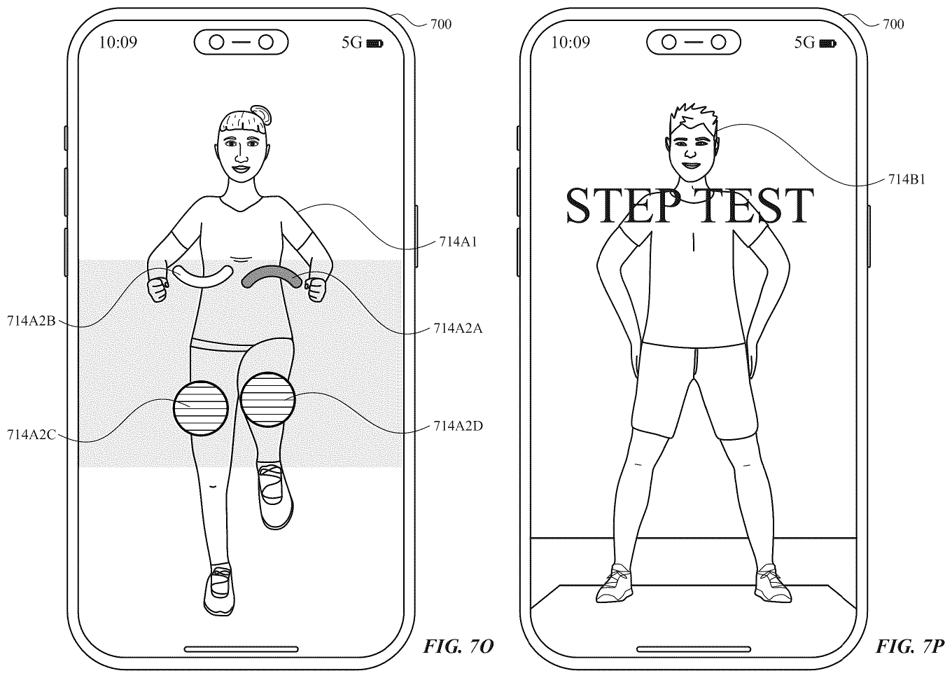

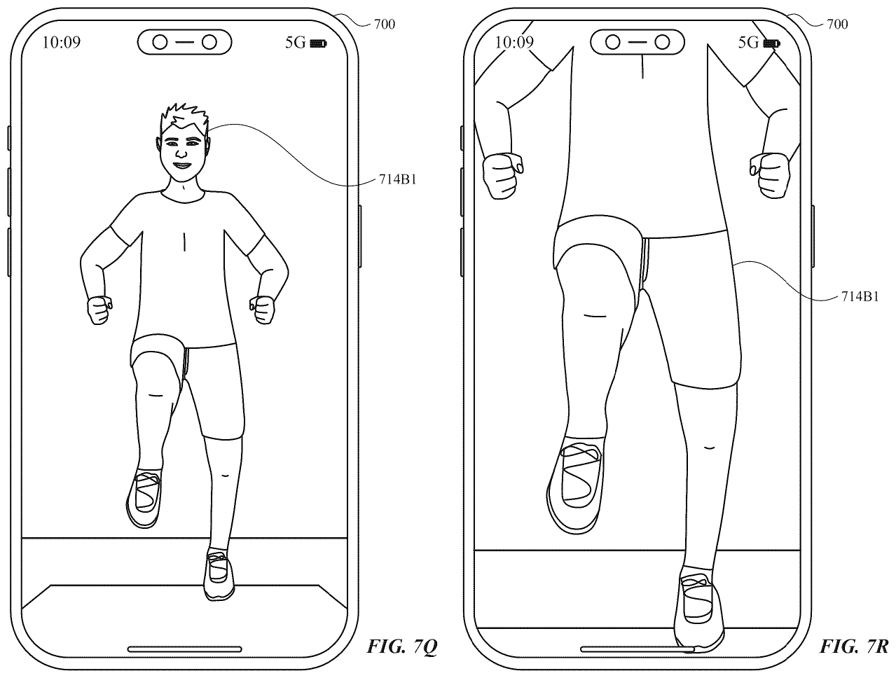

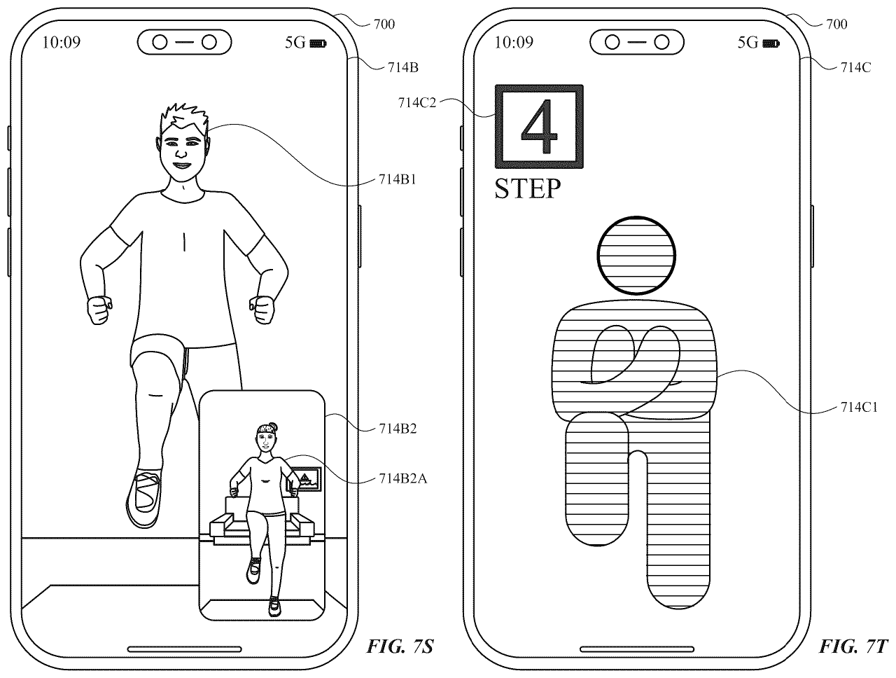

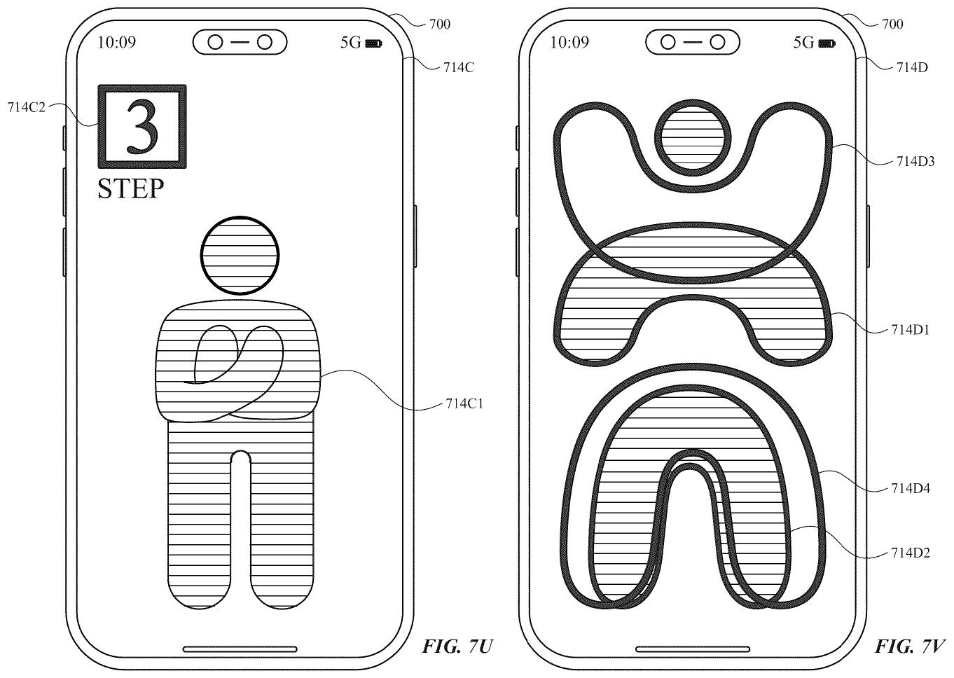

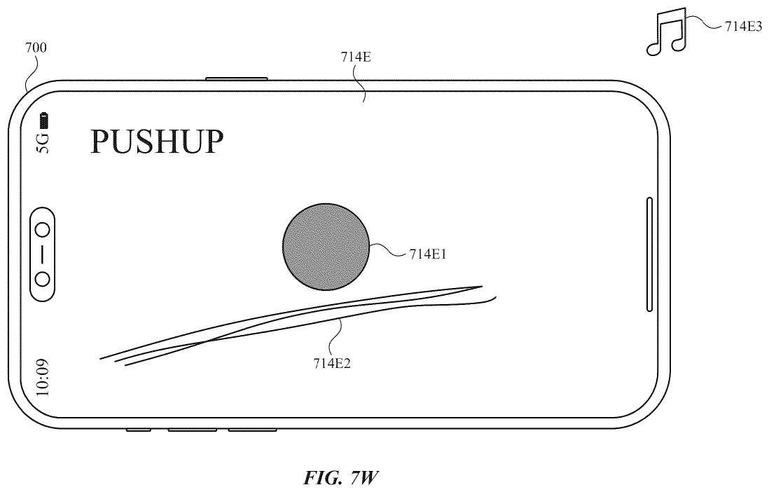

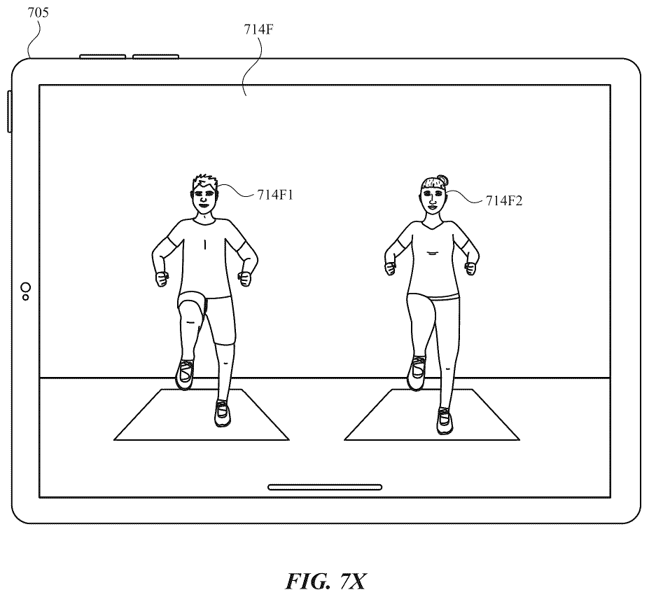

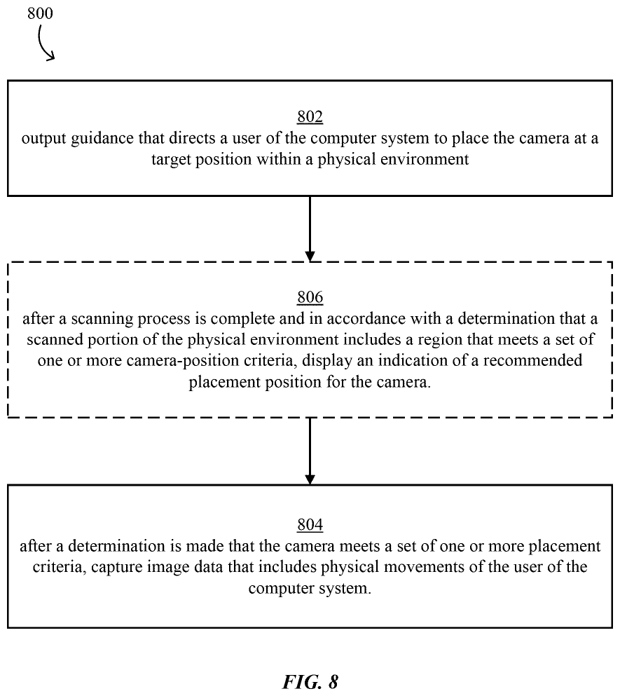

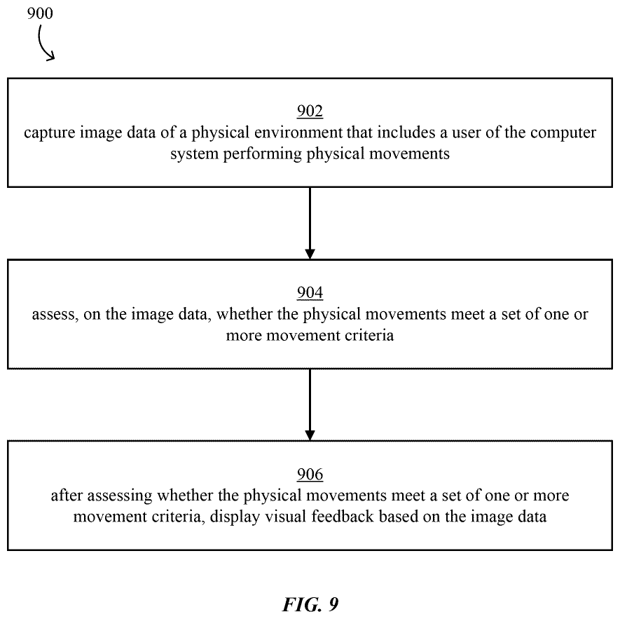

Below, FIGS. 1A-1B, 2, 3, 4A-4B, and 5A-5B provide a description of exemplary devices for performing the techniques for camera-based assessment of physical movement. FIG. 6 is top-down schematic of a physical environment of a user, in accordance with some embodiments. FIGS. 7A-7X illustrate exemplary user interfaces for camera placement and physical movement capture and also for camera-based assessment of physical movement using a computer system, in accordance with some embodiments. FIG. 8 is a flow diagram of methods for camera placement and physical movement capture, in accordance with some embodiments. FIG. 9 is a flow diagram of methods for camera-based assessment of physical movement using a computer system, in accordance with some embodiments. The schematic of FIG. 6 and the user interfaces in FIGS. 7A-7X are used to illustrate the processes described below, including the processes in FIGS. 8 and 9.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, reducing the amount of time required to properly position a camera, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

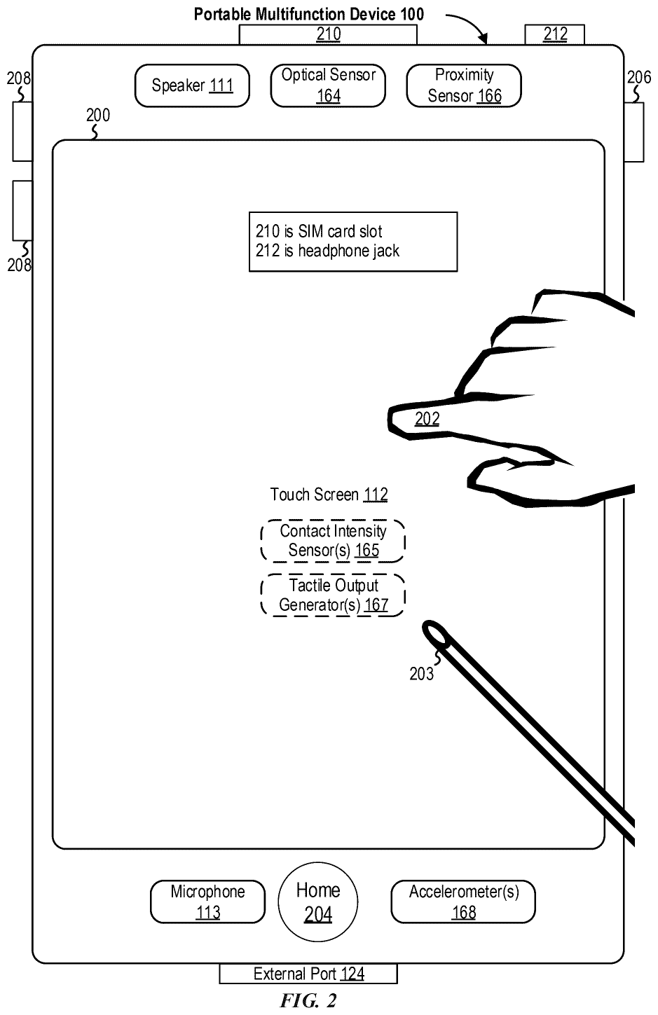

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both cars) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Patents: U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

In some embodiments, a depth map (e.g., depth map image) contains information (e.g., values) that relates to the distance of objects in a scene from a viewpoint (e.g., a camera, an optical sensor, a depth camera sensor). In one embodiment of a depth map, each depth pixel defines the position in the viewpoint's Z-axis where its corresponding two-dimensional pixel is located. In some embodiments, a depth map is composed of pixels wherein each pixel is defined by a value (e.g., 0-255). For example, the “0” value represents pixels that are located at the most distant place in a “three dimensional” scene and the “255” value represents pixels that are located closest to a viewpoint (e.g., a camera, an optical sensor, a depth camera sensor) in the “three dimensional” scene. In other embodiments, a depth map represents the distance between an object in a scene and the plane of the viewpoint. In some embodiments, the depth map includes information about the relative depth of various features of an object of interest in view of the depth camera (e.g., the relative depth of eyes, nose, mouth, ears of a user's face). In some embodiments, the depth map includes information that enables the device to determine contours of the object of interest in a z direction.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

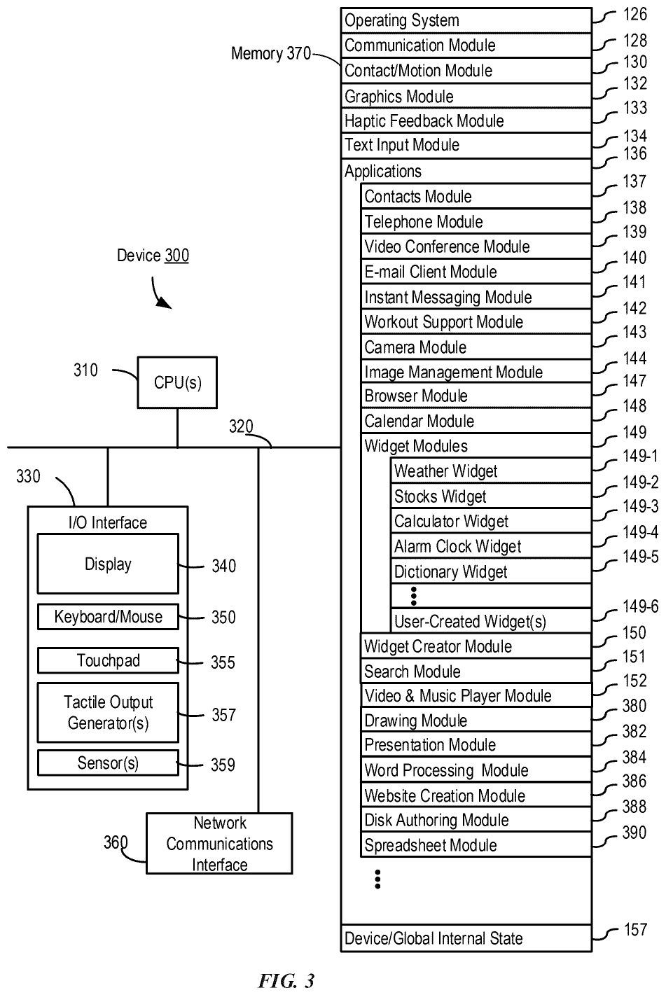

In some embodiments, the software components stored in memory 102 include operating system 126, communication module (or set of instructions) 128, contact/motion module (or set of instructions) 130, graphics module (or set of instructions) 132, text input module (or set of instructions) 134, Global Positioning System (GPS) module (or set of instructions) 135, and applications (or sets of instructions) 136. Furthermore, in some embodiments, memory 102 (FIG. 1A) or 370 (FIG. 3) stores device/global internal state 157, as shown in FIGS. 1A and 3. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, iOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3 is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3 is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Attention is now directed towards embodiments of user interfaces that are, optionally, implemented on, for example, portable multifunction device 100.

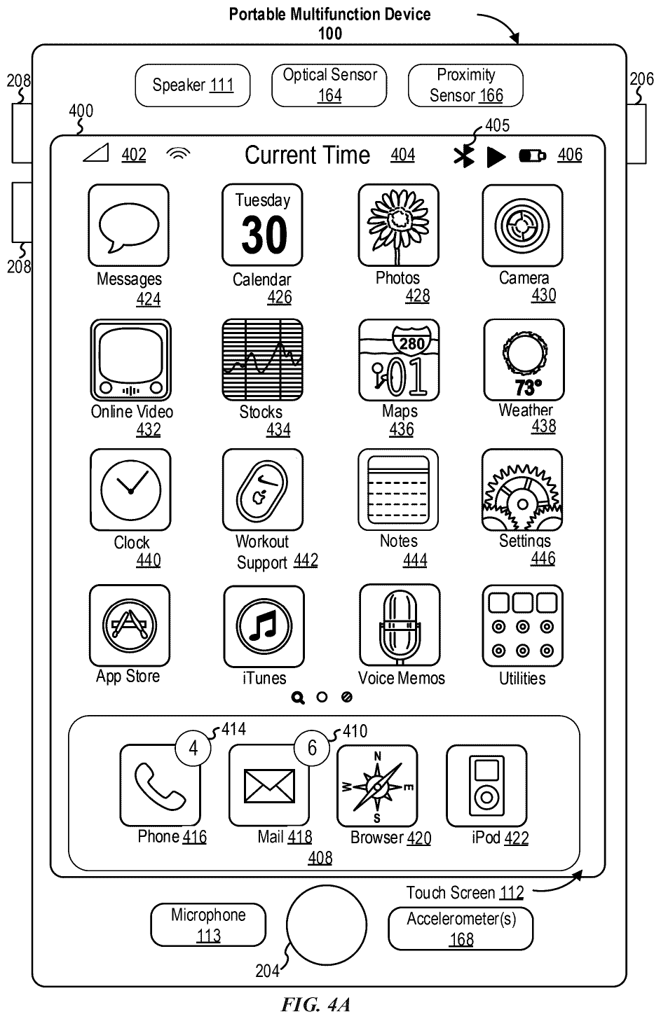

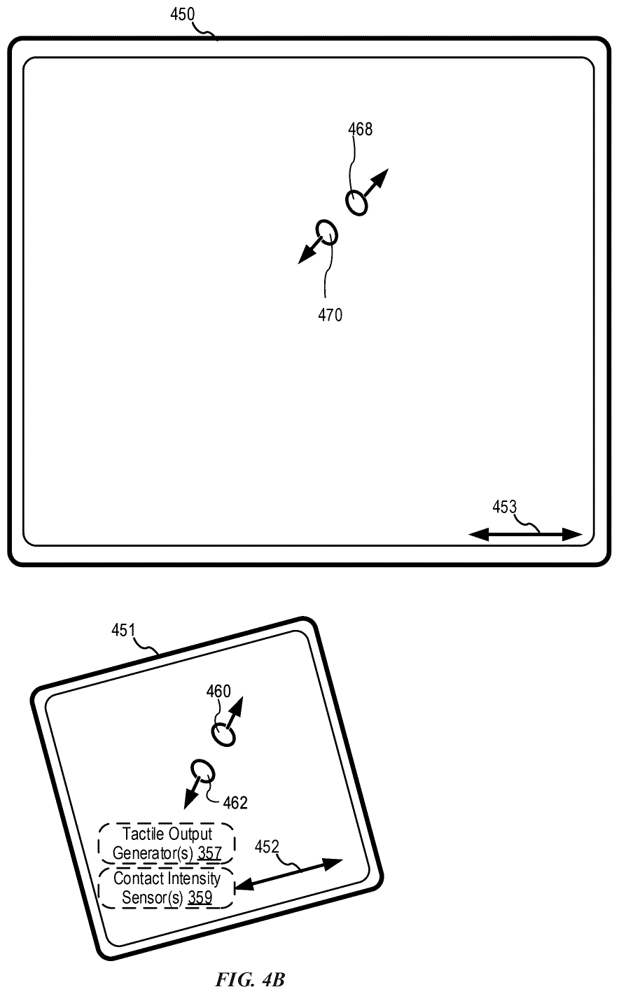

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3) with a touch-sensitive surface 451 (e.g., a tablet or touchpad 355, FIG. 3) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

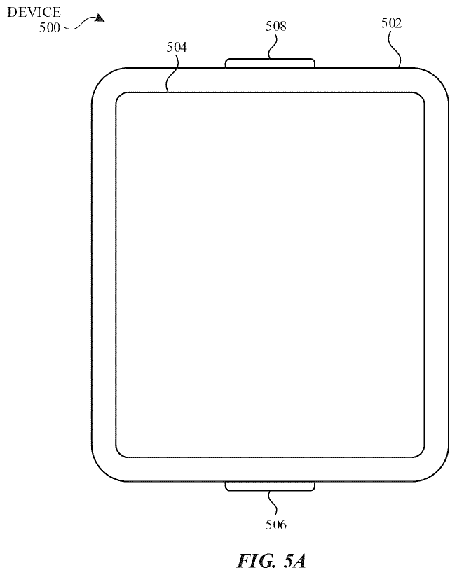

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3. Device 500 has bus 512 that operatively couples I/O section 514 with one or more computer processors 516 and memory 518. I/O section 514 can be connected to display 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including processes 800 and 900 (FIGS. 8 and 9, respectively). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3 or touch-sensitive surface 451 in FIG. 4B) while the cursor is over a particular user interface element (e.g., a button, window, slider, or other user interface clement), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

Attention is now directed towards embodiments of user interfaces (“UI”) and associated processes that are implemented on an electronic device, such as portable multifunction device 100, device 300, or device 500.

FIG. 6 is top-down schematic of a physical environment 600. At FIG. 6, user 604 is situated in room 602, which includes sofa 606 and table 608. Dashed area 605 denotes a location at which user 604 is standing within room 602. Hatched area 610 denotes areas of room 602 in which a camera cannot be placed and still have a field-of-view that is required and/or is optimal for capturing image data to assess the physical movements of user 604. For example, while the area to the right of sofa 606 would be sufficiently far from user 604, the view of user 604 would be obstructed by the sofa, so the camera should not be placed in that area. Unhatched area 612 denotes areas of room 602 where a camera can be placed so that the camera has a required and/or optimal field-of-view for capturing image data to assess the physical movements of user 604 (e.g., the camera would be at least a predetermined distance away (e.g., a distance of 4 feet, 5 feet, 6 feet, or a distance based on a characteristic of a user (e.g., a height or an arm span)) from the position of user 604 and would have an unobstructed view of user 604). In some embodiments, the predetermined distance varies depending on the nature of physical movements that will be assessed (e.g., certain exercises and/or activities can required different distances, perspectives, and/or orientations). Dashed area 614 denotes a specific sub-area (e.g., an area along a wall of room 602) within unhatched area 612 at which a camera can be placed. In some embodiments, dashed area 614 depicts an area that is determined to be the most optimal area for placement.

FIGS. 7A-7X illustrate exemplary user interfaces for camera placement and physical movement capture and assessment, in accordance with some embodiments. The user interfaces in these figures are used to illustrate the processes described below, including the processes in FIGS. 8 and 9.

FIG. 7A depicts device 700, which is a computing device being operated by user 604 (e.g., while user 604 is situated at dashed area 605). Device 700 includes display 702 (e.g., a touch-sensitive display capable of detecting touch inputs) and image sensors 704 (e.g., camera(s) (e.g., forward facing camera(s)) and/or depth sensor(s)). In some embodiments, device 700 includes additional rear-facing image sensors (e.g., camera(s) and/or depth sensor(s)) on the opposite side of display 702 that can capture image data with a different field-of-view than that of image sensors 704). In some embodiments, device 700 is a smart phone, a smart watch, a tablet computer, and/or a personal computer. In some embodiments, device 600 includes one or more features of devices 100, 300, and/or 500.

At FIG. 7A, device 700 displays user interface 706, which is a user interface of a health or fitness application (e.g., a health and/or fitness tracking, health monitoring, workout application, and/or health-data aggregating application). User interface 706 includes a notification 706A, reminding the user of device 700 to perform a physical movement assessment (e.g., as part of a recurring (e.g., annual) health assessment). In some embodiments, notification 706A is a reminder to perform a workout of an ongoing fitness plan. At FIG. 7A, device 700 detects input 708A (e.g., a tap input), corresponding to notification 706A. In some embodiments, input 708A, and/or other inputs described with reference to FIGS. 7A-7X, is a gaze-based input, actuation of a hardware button (e.g., while the user's focus is detected at notification 706A), a gesture, or a mouse click.

At FIG. 7B, in response to input 708A, device 700 displays camera-positioning user interface 710A that includes representation 710A1 of the field-of-view (e.g., a live camera view finder) of a rear facing camera of device 700. Representation 710A1 includes an image of sofa 606, as user 604 is currently operating device 700 while situated as shown in FIG. 6. Camera-positioning user interface 710A includes scanning affordance 710A2 that can be selected to initiate a process for scanning the physical environment to assist user 604 in position device 700 (e.g., scanning to determine potential placement positions for device 700 (e.g., for the cameras of device 700). In some embodiments, camera-positioning user interface 710A (e.g., and other camera-positioning user interfaces described herein) output audio guidance and/or guidance with respect to non-position-related capture conditions such lighting level, glare, and/or physical obstructions). At FIG. 7B, device 700 detects input 708B corresponding to scanning affordance 710A2.

At FIG. 7C, in response to input 708B, device 700 initiates a scanning process using a camera (e.g., a rear-facing camera) of device 700 and, optionally, one or more additional sensors (e.g., depth sensors, orientation sensors, and/or positioning sensors). As part of the scanning process, device 700 updates camera-positioning user interface 710A to include scanned region indicator 710A3 that graphically indicates areas of room 602 that have been already scanned. In some embodiments, scanned region indicator 710A3 is animatedly expanded as scanning progresses and additional areas of room 602 are scanned. At FIG. 7C, device 700 detects rotation of device 700 in a leftwards/counter-clockwise direction, as indicated by indicator 712A.

FIG. 7D depicts device 700 after user 604 has reoriented device 700 to face table 608 (e.g., the user has turned left/counter-clockwise while remaining situated at dashed area 605). Scanned region indicator 710A3 indicates that a portion, but not all, of room 602 in the current field-of-view of a camera of device 700 has been scanned. At FIG. 7C, device 700 detects further rotation of device 700 in a leftwards/counter-clockwise direction, as indicated by indicator 712B.

FIG. 7E depicts device 700 after user 604 has reoriented device 700 to face the portion of room 602 that includes dashed area 614 of FIG. 6 (e.g., towards the left in FIG. 6) (e.g., the user has turned left/counter-clockwise while remaining situated at dashed area 605). Scanned region indicator 710A3 of FIG. 7E indicates that all of the portion of room 602 that is visible on display 702 has been scanned (e.g., the hatched pattern covers the entire visible floor of room 602). Camera-positioning user interface 710A now also includes (e.g., because scanning of the current field-of-view is complete) unsuitable region indicator 710A4 that is overlayed on the portion of representation 710A1 that is not suitable for camera placement for assessing physical movement of user 604 while the user is situated at dashed area 605 (e.g., not suitable because it is too close and/or because the view would be obstructed from that position). At FIG. 7E, unsuitable region indicator 710A4 corresponds to the portion of hatched area 610 of FIG. 6 that is currently visible in representation 710A. At FIG. 7E, camera-positioning user interface 710A also includes recommended position indicator 710A5 that depicts a specific, recommended placement position for device 700 within a portion of representation 710A that is outside of the area indicated by unsuitable region indicator 710A4 (e.g., a currently-visible portion of unhatched area 612 of FIG. 6. Recommended position indicator 710A5 corresponds to dashed area 614 shown in FIG. 6. In some embodiments, recommended position indicator 710A5 also indicates an orientation (e.g., facing and/or tilt) at which device 700 should be placed.

FIG. 7F depicts device 700 displaying camera-positioning user interface 710A with representation 710A updated to show a field-of-view that includes the portions of room 602 that includes sofa 606 and the area that corresponds to dashed area 605 of FIG. 6 (e.g., user 604 is standing at a location that corresponds to dashed area 614 of FIG. 6 with a camera of device 700 oriented towards the center of room 602). At FIG. 7F, camera-positioning user interface 710A includes object recommended placement indicator 710A6 that indicates a recommend placement position for an accessory (e.g., a yoga mat). In some embodiments, a placement position for a mat is provided to assist the user with proper positioning (e.g., positioning that is fully within the FOV of a camera of device 700) while the user performs physical movements.

FIG. 7G depicts device 700 displaying camera-positioning user interface 710B (e.g., an alternate to or supplement to camera-positioning user interface 710A). In some embodiments, camera-positioning user interface 710B is displayed in response to input 708A of FIG. 7A (e.g., displayed instead of camera-positioning user interface 710A). In some embodiments, camera-positioning user interface 710B is displayed in response to a selection from a plurality of available camera-positioning user interfaces that are displayed in response to input 708A (e.g., device 700 can present users with options for camera-positioning user interfaces to use). Camera-positioning user interface 710B includes measure affordance 710B1 that, when selected, initiates a process for camera-based measurements. At FIG. 7G, device 700 detects input 710B1 (e.g., a tap) corresponding to measure affordance 710B1.

At FIG. 7H, in response to input 710B1, device 700 updates camera-positioning user interface 710B to include representation 710B2 of the field-of-view (e.g., a live camera view finder) of a rear facing camera of device 700. At FIG. 7H, user 604 is operating device 700 while oriented as shown in FIG. 6. Thus, sofa 606 is visible in representation 710B2. Camera-positioning user interface 710B also includes virtual measurement tape 710B3 that indicates (e.g., based on data from one or more cameras and/or depth sensors) a distance (e.g., 5 feet, 3 inches) to a detected object (e.g., a closest detected object), sofa 606. Camera-positioning user interface 710B also includes information 710B4 that indicates that at least 6 feet of distance is required for proper device/camera placement. In some embodiments, the required distance (e.g., 6 feet) is the same for all users. In some embodiments, the required distance is based on a physical characteristic of the user (e.g., a height or an arm span of the user). In such embodiments, the value of the physical characteristic can be an estimated value (e.g., based on other parameters of the user such as sex and/or age), an entered value, or a measured value (e.g., measured via one or more sensors of device 700). At FIG. 7H, because the distance to sofa 606 is less than the required distance, device 700 cannot be properly placed in the space shown in representation 710B2. At FIG. 7H, device 700 detects rotation of device 700 in a leftwards/counter-clockwise direction, as indicated by indicator 712C.

FIG. 7I depicts device 700 after user 604 has reoriented device 700 by 180 degrees to face the portion of room 602 that includes dashed area 614 of FIG. 6 (e.g., towards the left in FIG. 6) (e.g., the user has turned left/counter-clockwise while remaining situated at dashed area 605). Representation 710B2 now includes the wall of the room shown on the left of FIG. 6 and virtual measurement tape 710B3 indicates that the distance to the wall is 6 feet and 2 inches, which is greater than the required 6 feet. In some embodiments, camera-positioning user interface 710B provides a user with a guide for positioning based on a minimum distance, without requiring the user to scan a physical environment (e.g., a room).

FIG. 7J depicts device 700 displaying camera-positioning user interface 710C (e.g., an alternate to or supplement to camera-positioning user interface 710A and/or camera-positioning user interface 710B). In some embodiments, camera-positioning user interface 710C is displayed in response to input 708A of FIG. 7A (e.g., displayed instead of camera-positioning user interfaces 710A and/or 710B). In some embodiments, camera-positioning user interface 710C is displayed in response to a selection from a plurality of available camera-positioning user interfaces that are displayed in response to input 708A (e.g., device 700 can present users with options for camera-positioning user interfaces to use). Camera-positioning user interface 710C includes information 710C1 that directs the user to place device 700 four steps away from the user's current position, which is an approximate guide to the required distance. In some embodiments, the guidance is provided in approximate units because the distance given is conservative (e.g., if the average step is 2 feet in length, only 6 feet may be required with a guidance of “4 steps” providing a buffer). In some embodiments, the guidance is the same for all users. In some embodiments, the guidance is based on a physical characteristic of the user (e.g., a height, an arm span, or stride of the user). In such embodiments, the value of the physical characteristic can be an estimated value (e.g., based on other parameters of the user such as sex and/or age), an entered value, or a measured value (e.g., measured via one or more sensors of device 700). Note that camera-positioning user interface 710C does not include a representation of a field-of-view of any cameras of device 700. Camera-positioning user interface 710C includes found space affordance 710C2 that, when selected, indicates that the user has placed device 700 at an appropriate location.

FIG. 7K depicts device 700 along with device 701 (e.g., a device including one or more features of devices 100, 300, 500, and/or 700), which is a smart watch that is being worn by user 604 and that is in communication with (e.g., paired with) device 700. At FIG. 7K, device 701 displays, on display 703, camera-positioning user interface 710D (e.g., an alternate to or supplement to camera-positioning user interfaces 710A, 710B, and/or 710C). In some embodiments, camera-positioning user interface 710D is displayed on device 701 in response to input 708A of FIG. 7A (e.g., displayed instead of displaying camera-positioning user interfaces 710A, 710B, and/or 710C on device 700). In some embodiments, camera-positioning user interface 710D is displayed in response to a selection from a plurality of available camera-positioning user interfaces that are displayed in response to input 708A (e.g., device 700 can present users with options for camera-positioning user interfaces to use). Camera-positioning user interface 710D includes information 710D1 that is similar to information 710C1 and also includes found space affordance 710D2 that is similar to found space affordance 710C2. In some embodiments, information 710D1 is also provided audibly to the user, via one or more audio speakers of device 700 and/or 701. In such embodiments, the user can receive camera position guidance without having to reference a display of device 700 and/or 701.

FIG. 7L depicts device 700 displaying camera-positioning user interface 710E1 and device 701 displaying camera-positioning user interface 710E2. In some embodiments, camera-positioning user interfaces 710E1 and 710E2 are displayed in response to input 708A of FIG. 7A (e.g., displayed instead of camera-positioning user interfaces 710A, 710B, 710C, and/or 710D). In some embodiments, camera-positioning user interfaces 710E1 and 710E2 are displayed in response to a selection from a plurality of available camera-positioning user interfaces that are displayed in response to input 708A (e.g., device 700 can present users with options for camera-positioning user interfaces to use). In some embodiments, camera-positioning user interfaces 710E1 and 710E2 are displayed as supplements to (e.g., displayed in addition to displaying) camera-positioning user interfaces 710A, 710B, 710C, and/or 710D. Camera-positioning user interface 710E1 includes representation 710E1A of the field-of-view of a camera of device 700 and camera-positioning user interface 710E2, on device 701, includes representation 710E2A of the field-of-view of the same camera of device 700. Thus, camera-positioning user interfaces 710E1 and 710E2 allow a user to place device 700 at a location and then move to a location at which the user's body fits within the borders of representations 710E1A and 710E2A (e.g., the user is able to easily review that a selected position is properly situated within the field-of-view of a camera of device 700). Camera-positioning user interface 710E2 is especially useful when the camera of device 700 is a rear-facing camera and display 702 of device 700 is not visible to the user, once device 700 has been placed in a potential position.

FIG. 7M depicts device 700 displaying an assessment user interface 714A that is displayed while the user is performing a step assessment that includes the user alternatively raising her knees to about waist level. In some embodiments, the user is performing (and device 700 is assessing) a different type of physical movement such as push-ups, sit-ups, lunges, and/or squats. In some embodiments, the user is performing a fitness activity such as aerobics, yoga, and/or a dance routine that device 700 assesses. Device 700 displays assessment user interface 714A after device 700 has been placed (e.g., placed based on guidance from camera-positioning user interfaces 710A, 710B, 710C, 710D, 710E1, and/or 710E2 and/or placed at dashed area 614 of FIG. 6) and after user 604 has positioned herself at a location for performing physical movements (e.g., at dashed area 605 of FIG. 6). Assessment user interface 714A includes representation 714A1 of user 604, as seen in the field-of-view of a camera of device 700. At FIG. 7M, representation 714A1 is an augmented reality representation of the user, where the user's video representation is overlaid on a virtual background (e.g., a blank background), so that the user appears as a live-action cutout. In some embodiments, displaying the user as an augmented reality cutout can assist the user in focusing on the user's movements without being distracted by the background. In some embodiments, representation 714A1 is a full representation of the field-of-view of the camera, without the user being cutout from the background that is the physical environment. In some embodiments, assessment user interface 714A is displayed on (e.g., without assessment user interface 714A being displayed on device 700 or concurrently with it being displayed on device 700) a device other than device 700 that is in communication with device 700. In such embodiments, assessment user interface 714A can be displayed on device 701 (e.g., a smartwatch), a tablet computer, a personal computer, a smart TV, and/or a digital media player outputting to a TV. At FIG. 7M, assessment user interface 714A also includes assessment objects 714A2A, 714A2B, 714A2C, and 714A2D. Objects 714A2A and 714A2B provide the user with guidance as to a target movement (e.g., a target location to which the user should alternatively lift her knees) while Objects 714A2C and 714A2D indicate a current detected location of the user's knees and also provides guidance as to which part of the user's body should be moved to the positions indicated by objects 714A2A and 714A2B. In some embodiments, one or more of objects 714A2A, 714A2B, 714A2C, and 714A2D are animated and/or otherwise visually varied (e.g., changing in color, size, and/or shape) to provide guidance. In some embodiments, objects 714A2A, 714A2B, 714A2C, and 714A2D indicate previous movements of the user (e.g., so as to provide a replay effect that indicates to the user how a past movement was performed). At FIG. 7M, assessment user interface 714A also includes emphasized region 714A3, that indicates a focus area for the user's movements. In some embodiments, emphasized region 714A3 is animated as the user performs movements (e.g., shrinks as the distance between the user's moving knee and a target location decreases). In some embodiments, representation 714A1 is modified to correct for one or more image capture factors. For example, if device 700 is positioned (e.g., on the floor) in manner such that a field-of-view of the camera is angled upwards (e.g., at a 10 degree, 15 degree, 30 degree, or 45 degree angle), device 700 can employ one or more image correction techniques so that representation 714A1 presents a head-on view of the user. In some embodiments, device 700 displays representation 714A1 at a size that is larger or smaller than that captured by the one or more cameras (e.g., representation 714A1 is a zoomed in or zoomed out representation). At FIG. 7M, device 700 detects user 604 physically moving.

At FIG. 7N, in response to detecting the movement of user 604 (e.g., as represented by the new position of representation 714A1), device 700 animates and modifies the appearance of objects 714A2A, 714A2B, and 714A2C. For example, object 714A2B changes color to indicate that the user is currently lifting her right leg and changes shape to indicate a position to which the user should move/position her right knee. Object 714A2A has also changed color, to indicate that it is not the currently moving leg. Objects 714A2C and 714A2D have been moved, to match the current detected locations of the user's knees. At FIG. 7N, device 700 detects further physical movement of user 604.

At FIG. 7O, in response to detecting user 604 shifting to movement of her left leg (e.g., as represented by the new position of representation 714A1), device 700 further animates and modifies the appearance of objects 714A2A, 714A2B, and 714A2C.

FIG. 7P depicts device 700 displaying an assessment user interface 714B that is an alternative to assessment user interface 714A. In some embodiments, a user is presented with an option to select from a plurality of assessment user interface options. In some embodiments, a setting of device 700 can be modified to indicate which assessment user interface is displayed while device 700 assesses movements of the user. At FIG. 7P, assessment user interface 714B is displayed while the user is performing a step assessment that includes the user alternatively raising her knees to about waist level. Assessment user interface 714B includes representation 714B1 of a coach (e.g., a person other than user 604) that will perform movements to guide user 604 through the step assessment. In FIG. 7P, representation 714B1 does not include any camera data from a camera of device 700; rather, representation 714B1 is a pre-recorded video (in some embodiments, is a dynamic composite of pre-recorded segments of video) of the coach or a device-generated graphical object (e.g., a virtual coach). In some embodiments, representation 714B1 is part of an augmented reality experience, with the coach being overlaid on a background representation of the actual physical environment (e.g., room 602 of FIG. 6) that is based on camera data captured by device 700 (e.g., so as to make it appear as if the coach is in room 602). In some embodiments, representation 714B1 is composed, over time, of different segments of videos and/or animations of the coach that are selected and presented based on an assessment of the physical movement of the user. For example, if the user performs repetitions with different errors, different segments of videos and/or animations of the coach can be selected and played, to create a custom coaching experience.

FIG. 7Q depicts assessment user interface 714B at a later point in time during the step assessment. Representation 714B1 of the coach has moved to provide guidance on the movement that the user should perform. In the embodiment of FIG. 7Q, the movement of representation 714B1 of the coach shows an idealized or target movement that the user should attempt to copy. In some embodiments, the movement of representation 714B1 of the coach mirrors detected movement of the user. At FIG. 7Q, device 700 detects that the physical movement of the user does not match a target movement (e.g., the user is not lifting her knee high enough).

At FIG. 7R, device 700 magnifies a portion (e.g., a portion around the coach's right knee) of representation 714B1 of the coach to indicate to the user that correction of the user's movement is required. In some embodiments, representation 714B1 can be modified in other ways to emphasize a target movement (e.g., representation 714B1 can be rotated to show a different angle for the movement and/or one or more virtual objects can be displayed to indicate a focus area).

At FIG. 7S, device 700 displays assessment user interface 714B with self-view inset 714B2 that includes representation 714B2A of the user and that provides the user with a live view of the field-of-view of the camera of device 700. In some embodiments, representation 714B2A is part of an augmented reality representation, similar to representation 714A1 of assessment user interface 714A. In some embodiments, self-view inset 714B2 can be invoked or dismissed in response to user input. In some embodiments, self-view inset 714B2 is displayed or not displayed based on a device setting. In some embodiments, self-view inset 714B2 is displayed in response to the movements of the user meeting certain criteria (e.g., when the movements do not meet a target movement).

FIG. 7T depicts device 700 displaying an assessment user interface 714C that is an alternative to assessment user interfaces 714A and/or 714B. In some embodiments, a user is presented with an option to select from a plurality of assessment user interface options. In some embodiments, a setting of device 700 can be modified to indicate which assessment user interface is displayed while device 700 assesses movements of the user. At FIG. 7T, assessment user interface 714C is displayed while the user is performing a step assessment step that includes the user alternatively raising her knees to about waist level. Assessment user interface 714C includes representation 714C1 of the user. Representation 714C1 is a fully virtual avatar or puppet of the user that is animated based on movements of the user detected by device 700 (e.g., detected by a camera of device 700). In some embodiments, representation 714C1 is presented from a different perspective than the perspective of the user, as captured by the camera of device 700 (e.g., user 604 is captured head-on but representation 714C1 is presented from a side or backside view). Assessment user interface 714C also includes indicator 714C2 that provides a countdown (or a count up) of repetitions of the step movement that have been detected by device 700. In some embodiments, only repetitions that meet target criteria (e.g., repetitions where the user's knees are lifted high enough) are counted.

At FIG. 7U, the user has successfully completed another repetition of the step movement, so indicator 714C2 now shows a count of three more steps required to complete the assessment.

FIG. 7V depicts device 700 displaying an assessment user interface 714D that is an alternative to assessment user interfaces 714A, 714B, and/or 714C. In some embodiments, a user is presented with an option to select from a plurality of assessment user interface options. In some embodiments, a setting of device 700 can be modified to indicate which assessment user interface is displayed while device 700 assesses movements of the user. At FIG. 7V, assessment user interface 714D is displayed while the user is performing an arm lift assessment that includes the user raising her arms above her head, in unison. Assessment user interface 714D includes a two-part avatar that includes arm portion 714D1 and leg portion 714D2. The two-part avatar is animated based on movements of the user detected by device 700 (e.g., detected by a camera of device 700). Assessment user interface 714D also includes guidance silhouettes 714D1 and 714D2 that correspond to target positions for the user's arms and legs, respective. guidance silhouettes 714D1 and 714D2 animate and otherwise change to indicate a target movement. For example, as seen in FIG. 7V, the user should move in a manner that causes arm portion 714D1 to match guidance silhouette 714D1 while keeping her legs in a position that causes leg portion 714D2 of the two-part avatar to remain within guidance silhouette 714D2.

FIG. 7W depicts device 700 displaying an assessment user interface 714E that is an alternative to assessment user interfaces 714A, 714B, 714C, and/or 714D. In some embodiments, a user is presented with an option to select from a plurality of assessment user interface options. In some embodiments, a setting of device 700 can be modified to indicate which assessment user interface is displayed while device 700 assesses movements of the user. At FIG. 7W, assessment user interface 714E is displayed while the user is performing a push-up assessment. Assessment user interface 714E include indicators 714E1 and 714E2 that are both animated based on movements of the user detected by device 700 (e.g., detected by a camera of device 700). Indicator 714E1 animates to show an average movement of the user's body, as the user performs push-ups. Indicator 714E2 animates to show movement of the user's body and to show whether the user's body is being maintained in a sufficiently straight position, as required for a proper push-up. Device 700 also outputs audio output that varies (e.g., varies in pitch, tone, and/or volume) as the user performs physical movements and/or as the user completes a valid repetition of a target movement (e.g., completes a pushup). In some embodiments, assessment user interfaces 714A, 714B, 714C, and/or 714D also output similar audio.

FIG. 7X depicts device 705, a tablet computer (e.g., a device including one or more features of devices 100, 300, 500, 700 and/or 701), displaying an assessment user interface 714F that is an alternative to assessment user interfaces 714A, 714B, 714C, 714D, and/or 714E. In some embodiments, a user is presented with an option to select from a plurality of assessment user interface options. In some embodiments, a setting of device 700 can be modified to indicate which assessment user interface is displayed while device 700 assesses movements of the user. At FIG. 7X, assessment user interface 714F is displayed while the user is performing a step assessment that includes the user alternatively raising her knees to about waist level. In some embodiments, assessment user interface 714F is displayed on device 700 and/or a personal computer, a smart TV, and/or a digital media player outputting to a TV. In some embodiments, while assessment user interface 714F is shown on device 705, the camera capturing the data being shown in assessment user interface 714F is integrated into device 700, which is in communication with device 705. Assessment user interface 714F includes representation 714F1 of a coach that includes one or more properties of representation 714B1, discussed above, and also includes representation 714F2 of the user that includes one or more properties of representation 714A1. In some embodiments, assessment user interface 714F is an augmented reality experience with live representation 714F2 (e.g., that is based on camera data captured by device 705) being superimposed on prerecorded video and/or virtual content that includes representation 714F1 of a coach. In assessment user interface 714F, the movements of representation 714F1 of a coach of the coach are not based on movements of the user, whereas the movements of representation 714F2 of the user are based on detected movements of the user. In some embodiments, such as the embodiment of FIG. 7X, representation 714F1 and 714F2 are presented from the same perspective (e.g., head-on). In some embodiments, representation 714F1 and 714F2 are presented from different perspectives (e.g., representation 714F1 of the coach is presented head-on but representation 714F2 of the user is shown from behind such that it appears as if the user is in an exercise class setting). In some embodiments, representation 714F2 of the user is based on camera data captured by a device other than 705 (e.g., by a smart phone or smart watch of the user).

FIG. 8 is a flow diagram illustrating a method for camera placement and physical movement capture using a computer system in accordance with some embodiments. Method 700 is performed at a computer system (e.g., 100, 300, 500, 700, 701, and/or 705; e.g., a smart phone, a smart watch, a personal computer, a digital media player) that is in communication with a camera (e.g., 704 and/or a single camera or a plurality of cameras) (in some embodiments, the camera is integrated into a housing of the computer system; in some embodiments, the camera is separate from the computer system) and an output device (e.g., 702, 703, and/or a single output device or a plurality of output devices; a display generation component (e.g., a display (e.g., touch-sensitive display and/or a projector), an audio speaker, and/or a haptic generator) (in some embodiments, the camera and/or the output device are integrated into the computer system; in some embodiments, the camera and/or the output device are wirelessly connected to the computer system (e.g., the camera and/or output device are a separate device and/or integrated into an external device)). Some operations in method 800 are, optionally, combined, the orders of some operations are, optionally, changed, and some operations are, optionally, omitted.

In some embodiments, the electronic device (e.g., 700) is a computer system. The computer system is optionally in communication (e.g., wired communication, wireless communication) with a display generation component and with one or more input and/or output devices. The display generation component is configured to provide visual output, such as display via a CRT display, display via an LED display, or display via image projection. In some embodiments, the display generation component is integrated with the computer system. In some embodiments, the display generation component is separate from the computer system. The one or more input devices are configured to receive input, such as a touch-sensitive surface receiving user input. In some embodiments, the one or more input devices are integrated with the computer system. In some embodiments, the one or more input devices are separate from the computer system. Thus, the computer system can transmit, via a wired or wireless connection, data (e.g., image data or video data) to an integrated or external display generation component to visually produce the content (e.g., using a display device) and can receive, a wired or wireless connection, input from the one or more input devices.

As described below, method 800 provides an intuitive way for camera placement and physical movement capture. The method reduces the cognitive burden on a user for camera placement and physical movement capture, thereby creating a more efficient human-machine interface. For battery-operated computing devices, enabling a user to conduct a camera-based assessment of physical movement faster and more efficiently conserves power and increases the time between battery charges.

The computer system (e.g., 700, 701, 705) outputs (802), via the output device (e.g., 702, 703), guidance (e.g., 710A5, 710B4, 710B3, 710C1, 710D1) that directs a user of the computer system to place the camera (e.g., place the computer system when the computer system includes the camera) at a target position (e.g., 612, 614) within a physical environment (e.g., 602) (e.g., a space and/or room that include the user).

After (in some embodiments, in response to) a determination is made (e.g., by the computer system, by an external device (e.g., a server) that the camera (in some embodiments, the computer system; in some embodiments, the field-of-view of the camera) meets a set of one or more placement criteria (e.g., criteria of discussed with reference to FIGS. 7E, 7H, and 7J) (e.g., criteria regarding whether the placement and/or orientation of the camera is sufficient to capture movement of the user) (in some embodiments, determining whether the criteria are met includes generating and/or analyzing a depth map of the environment), the computer system captures (804), via the camera, image data (e.g., data captured during physical assessments of FIGS. 7M-7X) (e.g., still and/or video image data) that includes physical movements (e.g., detected and/or assessed movement) of the user of the computer system. Outputting guidance that directs a user of the computer system to place the camera at a target position provides improved feedback as to proper camera placement and reduces the amount of time required to properly position the camera and can also improve the quality of image data capture. Providing improved feedback and reducing the amount of time required to properly position the camera enhances the operability of the computer system and makes the user-system interface more efficient (e.g., by helping the user to provide proper inputs and reducing user mistakes when operating/interacting with the system) which, additionally, reduces power usage and improves battery life of the device by enabling the user to use the system more quickly and efficiently.

In some embodiments, the guidance includes an indication (e.g., 710A2, 710A3) (e.g., a graphical indication, an audio indication, and/or a textual indication) to scan at least a first portion of the physical environment (e.g., a room and/or area where the user is currently located) using the camera. Displaying guidance that includes an indication to scan at least a first portion of the physical environment provides improved feedback as to the actions needed to properly place the camera, which provides improved feedback and reduces the amount of time required to properly position the camera and can also improve the quality of image data capture (e.g., due to proper placement).

In some embodiments, in accordance with a determination that set of one or more scanning-completion criteria (in some embodiments, the criteria includes a criterion that is met when a determination is made that threshold amount of the physical environment has been scanned (e.g., 20%, 30%, 40%, 50%, 75%, or 90%; an amount that includes one or more portions of the physical environment that meet a set of environmental placement criteria (e.g., whether a suitable position for placing the camera has been detected)) are not met, the computer system continues to output the guidance (e.g., continues to output 710A3 and/or 710B3) (in some embodiments, continuing to output the guidance includes updating the guidance to indicate scanning progress); and in accordance with a determination that the set of one or more scanning completion criteria are met, the computer system ceases output of the guidance. Ceasing output of the guidance when completion criteria are met performs an operation when a set of conditions has been met without requiring further user input and reduces the number of inputs needed to complete the scanning process.

In some embodiments, after a scanning process is complete (e.g., after ceasing to output the guidance and/or after the scanning-completion criteria are met): in accordance with a determination that a scanned portion of the physical environment includes a region that meets a set of one or more camera-position criteria (e.g., a position in which the camera can be placed in order to capture image data suitable for assessing physical movements of the user), the computer system displays (806) (e.g., via display generation component) an indication of a recommended placement position for the camera (e.g., 710A5, 710B3) (in some embodiments, for the computer system). In some embodiments, the computer system displays a representation of the field-of-view of the camera and the indication of the recommended placement position for the camera is positioned within the representation of the field-of-view of the camera. Displaying an indication of a recommended placement position for the camera when criteria are met performs an operation when a set of conditions has been met without requiring further user input, reduces the number of inputs needed to complete the scanning process, and provides improved feedback and reduces the amount of time required to properly position the camera and can also improve the quality of image data capture.

In some embodiments, the determination that a scanned portion of the physical environment includes a region that meets a set of one or more camera-position criteria is made based on data collected (e.g., via the camera) during the scanning process (e.g., as discussed with reference to FIG. 7E). In some embodiments, data collected during the scanning process is used to determine one or more characteristics (e.g., size and/or geometry) of the physical environment and determine whether a suitable region in the environment can be used to place the camera. Employing a scanning process to determine proper camera placement provides improved feedback and also reduces the number of inputs needed to perform an operation (e.g., by requiring less inputs to map out the physical environment) and also improves the accuracy of the determination process.

In some embodiments, during the scanning process, the computer system outputs an indication (e.g., a visual map) of the scanned physical environment. In some embodiments, the indication is updated on an ongoing basis as the scanning process continues.

In some embodiments, the guidance includes: a representation of a field-of-view of the camera (e.g., 710B2) (e.g., a live preview) that includes a representation of a second portion of the physical environment; and an indication (e.g., 710B3) (e.g., a graphical indication) (in some embodiments, the indication is an augmented-reality object that is scaled based on the physical environment (e.g., an AR ruler and/or measuring tape) of a distance between a current position of the camera and a third portion of the physical environment (e.g., a potential placement position for the camera), wherein the indication is overlaid on the representation of the field-of-view of the camera. Displaying an indication of a distance between a current position of the camera and a third portion of the physical environment provides improved feedback for placement of the camera and reduces the amount of time required to properly position the camera and can also improve the quality of image data capture.

In some embodiments, the indication includes a threshold distance (e.g., 710B4) (e.g., a target distance for placement of the camera) that is based on a height of the user of the computer system (e.g., an estimated height, a measured height, and/or an inputted height). Displaying an indication of a threshold distance that is based on a height of the user of the computer system provides improved feedback for placement of the camera and reduces the amount of time required to properly position the camera and can also improve the quality of image data capture.

In some embodiments, the guidance includes an indicator (e.g., 710C1) (e.g., an indicator of a threshold value (e.g., “4 steps”) or a value range (e.g., “4-5 steps”)) that is based on a physical characteristic (e.g., height, arm span length, and/or steps/strides) of the user of the computer system. Displaying an indication that is based on a physical characteristic of the user provides improved feedback for placement of the camera and reduces the amount of time required to properly position the camera and can also improve the quality of image data capture.

In some embodiments, the physical characteristic is a stride of the user (e.g., 710C1) (e.g., a distance covered by a step of the user) (in some embodiments, the guidance is “place the camera 4 steps away”).

In some embodiments, the physical characteristic of the user of the computer system is an estimated physical characteristic (e.g., an estimated height, arm span length, or stride length (in some embodiments, the same estimate is used for all users of the computer system).

In some embodiments, in accordance with a determination that the physical characteristic (e.g., height or arm span length) of the user has first value (e.g., the user has a certain height (e.g., 6′)), the indicator is first indicator (e.g., “place the camera 6 feet away”; and in accordance with a determination that a physical characteristic of the user has second value (e.g., a height of 5′8″), the indicator is second indicator (e.g., place the camera 5′8″ away), different from the first indicator. Displaying an indication that varies based on a physical characteristic of the user provides improved feedback for placement of the camera and reduces the amount of time required to properly position the camera and can also improve the quality of image data capture.

In some embodiments, the computer system is in communication with an external electronic device (e.g., 701) (e.g., a smart watch, a wireless headphone, a head-mounted display); the external electronic device includes the output device (e.g., 703) (e.g., the output device is integrated into the external electronic device (e.g., the output device is a speaker of the external electronic device)); and outputting the guidance includes outputting the guidance via the external electronic device (e.g., 710D1). Outputting the guidance via the external electronic device provides improved feedback and reduces the amount of time required to properly position the camera (e.g., by allowing the user to receive guidance separate from the location of the camera) and can also improve the quality of image data capture.

In some embodiments, the output device is a display of the external electronic device (e.g., 703); and the guidance includes a representation of the field-of-view of the camera that is displayed on the display of the of the external electronic device (e.g., 710E2A) (e.g., data from the camera is transmitted to the external electronic device). Providing guidance that includes a representation of the field-of-view of the camera that is displayed on the display of the of the external electronic device provides improved feedback and reduces the amount of time required to properly position the camera (e.g., by providing feedback on the current FOV of the user) and can also improve the quality of image data capture.

In some embodiments, the output device is an audio speaker of the external electronic device; and the guidance includes audio guidance outputted by the audio speaker of the external electronic device. Providing guidance that includes audio guidance outputted by the audio speaker of the external electronic device provides improved feedback and reduces the amount of time required to properly position the camera (e.g., by allowing the user to receive guidance separate from the location of the camera) and can also improve the quality of image data capture.

In some embodiments, the set of one or more placement criteria includes a criterion that is based on a distance relative to a position of the camera when the guidance is outputted (e.g., 710B3). In some embodiments, the placement criteria is based on placement relative to a current position of the camera and/or computer system when the guidance is initially outputted. In some embodiments, the set of one or more placement criteria is met when the camera is within a target range of distances from the current position of the camera when the guidance is initially outputted. Including a criterion that is based on a distance relative to a position of the camera when the guidance is outputted reduces the amount of time required to properly position the camera and can also improve the quality of image data capture (e.g., due to proper placement).

In some embodiments, the computer system outputs, via the output device, object-placement guidance that directs the user of the computer system to place a first object (e.g., 710A6) (e.g., an exercise mat, an exercise accessory (e.g., weights and/or other exercise apparatus)) at a second target position within the physical environment. Outputting object-placement guidance that directs the user of the computer system to place a first object at a second target position within the physical environment provides improved feedback and also reduces the amount of time required to place the first object.

In some embodiments, the computer system is in communication with a display generation component (e.g., an integrated display, a head-mounted display, a wirelessly connected display), the method further comprising: after the determination is made that the camera meets the set of one or more placement criteria, displaying, via the display generation component: a second representation of the field-of-view of the camera that includes a representation of a fourth portion of the physical environment; and a positioning indicator that indicates a position within the fourth portion of the physical environment (e.g., the portion visible in the FOV of the camera) at which the user should be positioned when performing the physical movements (e.g., as seen in FIGS. 7F and 7L). Displaying an indicator of where the user should be positioned when performing physical movements provides improved feedback and improves the quality of image data capture (e.g., due to proper placement).

In some embodiments, the computer system outputs, via the output device, capture-condition guidance (graphical, textual, and/or audio guidance) that indicates one or more non-position-based conditions that affect image capture via the camera (e.g., as discussed with reference to FIG. 7B) (e.g., guidance regarding lighting, glare, and/or obstructions that affect image capture). Outputting guidance that indicates one or more non-position-based conditions that affect image capture via the camera provides improved feedback and improves the quality of image data capture (e.g., due to having proper conditions).

In some embodiments, the guidance includes guidance on placement of the camera within the physical environment and guidance on orientation (an angle and/or tilt for the camera so that the field-of-view of the camera is properly situated to capture the physical movements of the user) of the camera within the physical environment (e.g., as discussed with reference to FIG. 7E). Outputting guidance on placement of the camera within the physical environment and guidance on orientation of the camera provides improved feedback and improves the quality of image data capture (e.g., due to proper camera positioning).

In some embodiments, after capturing the image data that includes physical movements of the user, the computer system assesses, based on the image data, whether the physical movements meet a set of one or more movement criteria (e.g., as discussed with reference to FIGS. 7M to 7X and method 900) (e.g., movement assessment criteria that determines whether the movement meets respective standards and/or conditions) (in some embodiments, assessing based on one or more aspects of method 900).

Note that details of the processes described above with respect to method 800 (e.g., FIG. 8) are also applicable in an analogous manner to the methods described below. For example, method 900 optionally includes one or more of the characteristics of the various methods described above with reference to method 800. For example, prior to assessing physical movements in accordance with method 900, a camera can be placed according to the method 800. For brevity, these details are not repeated below.

FIG. 9 is a flow diagram illustrating a method for camera-based assessment of physical movement using a computer system in accordance with some embodiments. Method 900 is performed at a computer system (e.g., 100, 300, 500, 700, 701 and/or 705; e.g., a smart phone, a smart watch, a personal computer, a digital media player) that is in communication with a camera (e.g., a single camera or a plurality of cameras) (in some embodiments, the camera is integrated into a housing of the computer system; in some embodiments, the camera is separate from the computer system) and a display generation component (e.g., 702, 703) (e.g., a display (e.g., touch-sensitive display and/or a projector) (in some embodiments, the camera and/or the display are integrated into the computer system; in some embodiments, the camera and/or the display generation component are wirelessly connected to the computer system (e.g., the camera and/or display generation component are a separate device and/or integrated into an external device)). Some operations in method 900 are, optionally, combined, the orders of some operations are, optionally, changed, and some operations are, optionally, omitted.

In some embodiments, the electronic device (e.g., 700) is a computer system. The computer system is optionally in communication (e.g., wired communication, wireless communication) with a display generation component and with one or more input and/or output devices. The display generation component is configured to provide visual output, such as display via a CRT display, display via an LED display, or display via image projection. In some embodiments, the display generation component is integrated with the computer system. In some embodiments, the display generation component is separate from the computer system. The one or more input devices are configured to receive input, such as a touch-sensitive surface receiving user input. In some embodiments, the one or more input devices are integrated with the computer system. In some embodiments, the one or more input devices are separate from the computer system. Thus, the computer system can transmit, via a wired or wireless connection, data (e.g., image data or video data) to an integrated or external display generation component to visually produce the content (e.g., using a display device) and can receive, a wired or wireless connection, input from the one or more input devices.

As described below, method 900 provides an intuitive way for camera placement and physical movement capture. The method reduces the cognitive burden on a user for camera placement and physical movement capture, thereby creating a more efficient human-machine interface. For battery-operated computing devices, enabling a user to conduct a camera-based assessment of physical movement faster and more efficiently conserves power and increases the time between battery charges.

The computer system (e.g., 700, 701, 705) captures (902), via the camera (e.g., 704), image data (e.g., still and/or video image data; in some embodiments data captured over a period of time) of a physical environment (e.g., a space and/or room that include the user) that includes a user of the computer system performing physical movements (e.g., as discussed with reference to FIGS. 7M-7X).

The computer system assesses (904), based on the image data, whether the physical movements meet a set of one or more movement criteria (e.g., as discussed with reference to FIGS. 7M-7X) (e.g., movement assessment criteria that determines whether the movement meets respective standards and/or conditions).

After (in some embodiments, in response to) assessing (e.g., determining) whether the physical movements meet a set of one or more movement criteria, the computer system displays (906), via the display generation component, visual feedback (e.g., one or more user interface objects) based on the image data (e.g., 714A2A-D, 714C2) (in some embodiments, based on the assessment of the physical movements). Providing visual feedback based on captured image data that includes physical movements of a user that is also assessed according to a set of criteria provides improved feedback to the user as to physical movements that the user has performed and assists the user with more properly performing additional movements.

In some embodiments, displaying the visual feedback includes displaying a virtual avatar (e.g., 714C1, 714D1-2, 714E1-2) (e.g., graphical puppet) that is animated based on the physical movements of the user captured in the image data. In some embodiments, the virtual avatar moves in accordance with the movements of the user. In some embodiments, the virtual avatar is a mirrored image of the user. Displaying a virtual avatar that is animated based on the physical movements of the user captured in the image data provides improved feedback to the user as to the physical movements captured in the image data assists the user with more properly performing additional movements.

In some embodiments, the virtual avatar is displayed without displaying a photorealistic representation of the user that is based on the captured image data (e.g., 714C1, 714D1-2, 714E1-2). In some embodiments, the virtual avatar is a stylized and/or abstracted representation of the user. Displaying a virtual avatar without displaying a photorealistic representation of the user provides the user with improved visual feedback that focuses in on movement of the avatar with distracting the user with their own representation, which, in some cases, can cause anxiety (e.g., due to body image issues).

In some embodiments, data corresponding to the user of the computer system in the captured image data includes data of the user of the computer system captured from a first perspective (e.g., a front-facing view of the user, an angled-up perspective of the front of the user, or a sideview of the user); and displaying the virtual avatar includes displaying the virtual avatar from a second perspective, different from the first perspective (e.g., as discussed with reference to FIG. 7M) (e.g., the virtual avatar is displayed from a different viewing angle than the capture angle of the user, as seen from the camera). Displaying the virtual avatar from a perspective other than that captured for the user can provide improved visual feedback (e.g., by providing a perspective that conveys improved feedback) and assists the user with more properly performing additional movements.

In some embodiments, data corresponding to the user of the computer system in the captured image data includes data of the user of the computer system captured at a first size within the field-of-view of the camera (e.g., the user occupies 25%, 40%, 50%, or 60% of FOV of the camera); and displaying the virtual avatar includes displaying the virtual avatar at a second size, different from the first size (e.g., as discussed with reference to FIG. 7M) (e.g., a size relative to the total displayed content area (e.g., 20%, 45%, 55%, or 70% of size of the total displayed content)). Displaying the virtual avatar with a different size than that captured for the user can provide improved visual feedback (e.g., by providing an avatar that conveys improved feedback) and assists the user with more properly performing additional movements.

In some embodiments, displaying the virtual avatar includes: in accordance with a determination that the physical movements meet the set of one or more movement criteria, displaying the virtual avatar in a first manner (in some embodiments, the first manner is an uncropped manner where the entirety of the avatar is represented) that includes displaying a portion of the virtual avatar at a third size, wherein the portion of the virtual avatar corresponds to a portion of the user of the computer system (e.g., a joint of the user (e.g., a knee or elbow that is involved in the assessed movement)) that is included in the captured image data; and in accordance with a determination that the physical movements do not meet the set of one or more movement criteria, displaying the virtual avatar in a second manner (in some embodiments, a cropped manner, where the entirety of the avatar is not represented), different than the first manner, that includes displaying the portion of the virtual avatar at a fourth size that is larger than the third size (e.g., as discussed with reference to FIG. 7Q-7R). In some embodiments, the avatar is zoomed in to emphasize a portion of the avatar that corresponds to the assessed movement (e.g., a knee(s) of the user is zoomed in when assessing a step test where the user alternates lifting his or her legs). Displaying a virtual avatar with a zoomed in focus on a portion of the avatar based on the assessment of the user's physical movements provides improved visual feedback on portions relevant to the assessment and also assists the user with more properly performing additional movements.

In some embodiments, the virtual avatar is displayed without displaying any direct representations (e.g., video and/or photo imagery) of the physical environment and/or of the user (e.g., the virtual avatar is displayed on a virtual background).

In some embodiments, displaying the visual feedback includes displaying an augmented reality environment that includes one or more virtual objects (e.g., 714A2A-D), wherein the one or more virtual objects are animated to provide guidance on a first set of target movements that should be performed by the user of the computer system (e.g., movements for a physical assessment and/or an exercise routine). Displaying visual feedback that includes an augmented reality environment that includes one or more virtual objects provides improved visual feedback on portions relevant to the assessment and also assists the user with more properly performing additional movements.

In some embodiments, displaying the augmented reality environment includes displaying a representation (e.g., a photorealistic representation, an isolated portion of the video data identified as being the user) of at least a portion of the captured image data that corresponds to the user of the computer system overlaid on a virtual background (e.g., 714A1 as shown in FIG. 7M) (e.g., at least a portion of the physical environment within the captured image data is not directly represented/presented). In some embodiments, video data corresponding to the user is identified in the image data and presented separately from the environmental image data, as part of an augmented reality environment. Displaying a representation of at least a portion of the captured image data that corresponds to the user of the computer system overlaid on a virtual background provides improved visual feedback on portions relevant to the assessment and also assists the user with more properly performing additional movements.

In some embodiments, data corresponding to the user of the computer system in the captured image data includes data of the user of the computer system captured from a first perspective (e.g., a front-facing view of the user, an angled-up perspective of the front of the user, or a sideview of the user); and displaying the augmented reality environment includes displaying the augmented reality environment from a second perspective, different from the first perspective (e.g., as discussed with reference to FIG. 7X) (e.g., the virtual objects and/or other portions of the environment are displayed from a different viewing angle than the capture angle of the user, as seen from the camera, so as to correct for a perspective of the camera). Displaying the augmented reality environment from a second perspective, different from the perspective captured of the user can provide improved visual feedback (e.g., by providing a perspective that conveys improved feedback) and assists the user with more properly performing additional movements.

In some embodiments, displaying the visual feedback includes displaying a video representation (in some embodiments, a non-virtual, photorealistic representation) of a first individual (e.g., 714B1) other than the user of the computer system, wherein the video representation includes the first individual performing a second set of target movements that should be performed by the user of the computer system (e.g., movements for a physical assessment and/or an exercise routine). Displaying a video representation that includes the first individual performing a second set of target movements that should be performed by the user of the computer system provides improved feedback to the user as to the physical movements captured in the image data assists the user with more properly performing additional movements.

In some embodiments, displaying the video representation of the first individual (e.g., 714B1) includes: prior to assessing whether the physical movements meet the set of one or more movement criteria, displaying a first portion of the video representation of the first individual; and after assessing the whether the physical movements meet the set of one or more movement criteria, displaying a second portion of the video representation of the first individual, wherein: in accordance with a determination that the physical movements meet the set of one or more movement criteria, the second portion of the video representation includes a first representation of the first individual (e.g., a default representation and/or a representation of the first individual continuing to perform a target movement); and in accordance with a determination that the physical movements do not meet the set of one or more movement criteria, the second portion of the video representation includes a second representation of the first individual that is different from the first representation (e.g., as discussed with reference to FIG. 7P) (e.g., the second representation includes first individual providing guidance on how the user of the computer system should adjust his or her movements and/or includes a zoomed in representation that focuses on a specific part of the first individual's body to improve the visibility of a target movement (e.g., a zoom in on the first individual's legs)). Displaying a second portion of the video representation of the first individual that varies based on whether the physical movements of the user meet the criteria performs an operation when a set of conditions has been met without requiring further user input and provides improved feedback to the user as to target physical movements and assists the user with more properly performing additional movements.

In some embodiments, while displaying the video representation of the first individual, the computer system displays a video representation of the user of the computer system based on the captured image data (e.g., 714B2A, 714F2) (e.g., picture-in-picture representation of captured image data that includes the user or an isolated portion of the video data identified as being the user that has been segmented from other portions of the video data and displayed next to the representation of the first individual (e.g., so the first individual and the user are presented, side-by-side)). Displaying the video representation of the first individual concurrently with displaying a video representation of the user of the computer system based on the captured image data provides improved feedback to the user as to the physical movements captured in the image data as well as provides improved feedback to the user as to target physical movements and assists the user with more properly performing additional movements and assists the user with more properly performing additional movements.

In some embodiments, the video representation of the first individual is displayed without displaying any video image data captured by the camera (e.g., without any direct feed captured from the camera).

In some embodiments, the physical movements correspond to a first repetition of a set of physical movements; and displaying the visual feedback includes displaying guidance (e.g., 714D3-4) on a second set of target movements that should be performed by the user of the computer system, wherein the guidance on the second set of target movements is based on an assessment of the first repetition of the set of physical movements. In some embodiments, after assessing a first representation of a target movement (e.g., a pushup), guidance is provided that is based on the assessed movement. Displaying guidance on a second set of target movements that should be performed by the user of the computer system based on the assessed physical movements provides improved feedback to the user as to the physical movements captured in the image data as well as provides improved feedback to the user as to target physical movements and assists the user with more properly performing additional movements and assists the user with more properly performing additional movements.

In some embodiments, the guidance on the second set of target movements does not include a representation of a human (e.g., 714D3-4) (e.g., does not include a video of the user or the first individual or a humanoid avatar). In some embodiments, the guidance on the second set of target movements includes one or more non-humanoid graphical objects (e.g., abstract, geometric shapes) that animated to provide guidance on a target movement.

In some embodiments, the guidance on the second set of target movements includes graphical content and audio content (e.g., 714D3-4) (e.g., speech guidance and a video guidance). Providing audio and graphical feedback as to target movements provides improved feedback to the user as to target physical movements and assists the user with more properly performing additional movements and assists the user with more properly performing additional movements.

In some embodiments, the guidance on the second set of target movements includes content that is responsive to ongoing physical movements of the user of the computer system that are captured by the camera (e.g., 714D3-4). In some embodiments, the guidance includes one or more graphical elements that animated based on tracked movements of the user. Providing guidance that is responsive to ongoing movements provides improved feedback to the user as to the physical movements captured in the image data as well as provides improved feedback to the user as to target physical movements and assists the user with more properly performing additional movements and assists the user with more properly performing additional movements.

In some embodiments, in accordance with a determination that the physical movements meet a second set of one or more movement criteria (in some embodiments, the second set of one or more movement criteria are met when the set of one or more movement criteria are not met (e.g., the second set of one or more movement criteria are met when the user's movement do not meet certain requirements, prompting further guidance)), the computer system displays, via the display generation component, guidance on a third set of target movements that should be performed by the user of the computer system (e.g., 714B1 of FIG. 7R); and in accordance with a determination that the physical movements do not meet the second set of one or more movement criteria, the computer system forgoes displaying the guidance on the third set of target movements that should be performed by the user of the computer system (in some embodiments, additional guidance is displayed only when the user's movements are not a qualifying movement for a required set of movements). Conditionally providing guidance when criteria are met or not met performs an operation when a set of conditions has been met without requiring further user input and also provides improved feedback and assists the user with more properly performing additional movements.

In some embodiments, displaying the visual feedback occurs after completing a process of image data capture that includes the capture of the image data and the assessing of whether the physical movements meet a set of one or more movement criteria (e.g., the visual feedback is a review (e.g., a recap) of the user's physical movements during the process of image data capture). Displaying feedback after completing the capture and assessment process provides the user with improved feedback at a time where the user is not distracted by performing actions and assists the user with more properly performing additional movements (e.g., during future assessments).

In some embodiments, the visual feedback is displayed over a representation of the user performing a physical movement that does not meet the set of one or more movement criteria (e.g., as seen in FIG. 7N). In some embodiments, the feedback is overlaid over representation of user to show an error (e.g., a failed pushup). Displaying visual feedback that is displayed over a representation of the user performing a physical movement that does not meet the set of one or more movement criteria performs an operation when a set of conditions has been met without requiring further user input and also provides improved feedback and assists the user with more properly performing additional movements.

In some embodiments, the camera is contained within a housing of a first electronic device (e.g., the computer system or a device in communication with the computer system) and the display generation component is in a housing of a second electronic device (e.g., the computer system or a device in communication with the computer system) that is different from and separate from the first electronic device (e.g., as discussed with reference to FIG. 7X). In some embodiments, the camera is in a smart phone and the display generation component is in a smart watch or a digital media player that is in communication with the smart phone. Providing feedback in a display generation component that is separate from the camera provides improved feedback that is not restricted to the location of the camera.

In some embodiments, while capturing the image data: in accordance with a determination that a set of one or more image capture correction criteria (in some embodiments, the criteria includes a criterion based on whether the user is properly positioned within the FOV of the camera, whether lighting conditions are correct, and/or whether the FOV of the camera is obstructed) are met, the computer system displays, via the display generation component, guidance for adjusting image capture (e.g., guidance on repositioning the camera and/or the user's position relative to the camera, guidance on improving lighting, and/or guidance on removing an obstruction); and in accordance with a determination that the set of one or more image capture correction criteria are not met, forgoing displaying the guidance for adjusting image capture. Conditionally providing guidance for adjusting the image capture performs an operation when a set of conditions has been met without requiring further user input and provides improved feedback to the user for improving image capture.

In some embodiments, while capturing the image data, the computer system displays an indication (e.g., 714C2) of a number of repetitions of physical movements that meet the set of one or more movement criteria (e.g., an indication of the number of instances of valid repetitions of a target movement (e.g., valid (e.g., having the proper form) push-ups, sit-ups, or squats)). Displaying an indication of repetitions that have met the criteria performs an operation when a set of conditions has been met without requiring further user input and provides improved feedback as to a number of qualifying repetitions.

In some embodiments, the computer system is packaged in a container (e.g., for distribution and/or for sale) in a package and/or container that also includes a collapsed stand (e.g., a plastic, cardboard, and/or metal stand) that can be used to position the camera in a proper position for capture of physical movements of the user. In some embodiments, custom accessories (e.g., weights) that are of known shape and/or include a recognizable pattern (e.g., motion capture-type dots) are provided (e.g., together with the computer system or separately) that can be used as part of an assessment of a user's physical movements.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve camera-based assessment of physical movement. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used in a method for camera-based assessment of physical movement. Accordingly, use of such personal information data enables users to have calculated control of the assessed movements. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of camera-based assessment of physical movement, the present technology can be configured to allow users to select to “opt in” or “opt out” of participation in the collection of personal information data during registration for services or anytime thereafter. In another example, users can select not to provide physical and/or biometric data for targeted assessment. In yet another example, users can select to limit the length of time such data is maintained or entirely prohibit the development of a physical and/or biometric profile of the user. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, assessments can be performed by inferring preferences and/or characteristics based on non-personal information data or a bare minimum amount of personal information, such as the content being requested by the device associated with a user, other non-personal information available to the computer system, or publicly available information.

What is claimed is:

1. A computer system configured to communicate with a camera and a display generation component, comprising:
one or more processors; and
memory storing one or more programs configured to be executed by the one or more processors, the one or more programs including instructions for:
capturing, via the camera, image data of a physical environment that includes a user of the computer system performing physical movements;
assessing, based on the image data, whether the physical movements meet a set of one or more movement criteria; and
after assessing whether the physical movements meet a set of one or more movement criteria, displaying, via the display generation component, visual feedback based on the image data.

2. The computer system of claim 1, wherein displaying the visual feedback includes displaying a virtual avatar that is animated based on the physical movements of the user captured in the image data.

3. The computer system of claim 2, wherein the virtual avatar is displayed without displaying a photorealistic representation of the user that is based on the captured image data.

4. The computer system of claim 2, wherein:
data corresponding to the user of the computer system in the captured image data includes data of the user of the computer system captured from a first perspective; and
displaying the virtual avatar includes displaying the virtual avatar from a second perspective, different from the first perspective.

5. The computer system of claim 2, wherein:
data corresponding to the user of the computer system in the captured image data includes data of the user of the computer system captured at a first size within the field-of-view of the camera; and
displaying the virtual avatar includes displaying the virtual avatar at a second size, different from the first size.

6. The computer system of claim 5, wherein displaying the virtual avatar includes:
in accordance with a determination that the physical movements meet the set of one or more movement criteria, displaying the virtual avatar in a first manner that includes displaying a portion of the virtual avatar at a third size, wherein the portion of the virtual avatar corresponds to a portion of the user of the computer system that is included in the captured image data; and
in accordance with a determination that the physical movements do not meet the set of one or more movement criteria, displaying the virtual avatar in a second manner, different than the first manner, that includes displaying the portion of the virtual avatar at a fourth size that is larger than the third size.

7. The computer system of claim 1, wherein displaying the visual feedback includes displaying an augmented reality environment that includes one or more virtual objects, wherein the one or more virtual objects are animated to provide guidance on a first set of target movements that should be performed by the user of the computer system.

8. The computer system of claim 7, wherein displaying the augmented reality environment includes displaying a representation of at least a portion of the captured image data that corresponds to the user of the computer system overlaid on a virtual background.

9. The computer system of claim 7, wherein:
data corresponding to the user of the computer system in the captured image data includes data of the user of the computer system captured from a first perspective; and
displaying the augmented reality environment includes displaying the augmented reality environment from a second perspective, different from the first perspective.

10. The computer system of claim 1, wherein displaying the visual feedback includes displaying a video representation of a first individual other than the user of the computer system, wherein the video representation includes the first individual performing a second set of target movements that should be performed by the user of the computer system.

11. The computer system of claim 10, wherein displaying the video representation of the first individual includes:
prior to assessing whether the physical movements meet the set of one or more movement criteria, displaying a first portion of the video representation of the first individual; and
after assessing the whether the physical movements meet the set of one or more movement criteria, displaying a second portion of the video representation of the first individual, wherein:
in accordance with a determination that the physical movements meet the set of one or more movement criteria, the second portion of the video representation includes a first representation of the first individual; and
in accordance with a determination that the physical movements do not meet the set of one or more movement criteria, the second portion of the video representation includes a second representation of the first individual that is different from the first representation.

12. The computer system of claim 10, the one or more programs further including instructions for:
while displaying the video representation of the first individual, displaying a video representation of the user of the computer system based on the captured image data.

13. The computer system of claim 1, wherein:
the physical movements correspond to a first repetition of a set of physical movements; and
displaying the visual feedback includes displaying guidance on a second set of target movements that should be performed by the user of the computer system, wherein the guidance on the second set of target movements is based on an assessment of the first repetition of the set of physical movements.

14. The computer system of claim 13, wherein the guidance on the second set of target movements does not include a representation of a human.

15. The computer system of claim 13, wherein the guidance on the second set of target movements includes graphical content and audio content.

16. The computer system of claim 13, wherein the guidance on the second set of target movements includes content that is responsive to ongoing physical movements of the user of the computer system that are captured by the camera.

17. The computer system of claim 1, the one or more programs further including instructions for:
in accordance with a determination that the physical movements meet a second set of one or more movement criteria, displaying, via the display generation component, guidance on a third set of target movements that should be performed by the user of the computer system; and
in accordance with a determination that the physical movements do not meet the second set of one or more movement criteria, forgoing displaying the guidance on the third set of target movements that should be performed by the user of the computer system.

18. The computer system of claim 1, wherein displaying the visual feedback occurs after completing a process of image data capture that includes the capture of the image data and the assessing of whether the physical movements meet a set of one or more movement criteria.

19. The computer system of claim 18, wherein the visual feedback is displayed over a representation of the user performing a physical movement that does not meet the set of one or more movement criteria.

20. The computer system of claim 1, wherein the camera is contained within a housing of a first electronic device and the display generation component is in a housing of a second electronic device that is different from and separate from the first electronic device.

21. The computer system of claim 1, the one or more programs further including instructions for:
while capturing the image data:
in accordance with a determination that a set of one or more image capture correction criteria are met, displaying, via the display generation component, guidance for adjusting image capture; and
in accordance with a determination that the set of one or more image capture correction criteria are not met, forgoing displaying the guidance for adjusting image capture.

22. The computer system of claim 1, the one or more programs further including instructions for:
while capturing the image data, displaying an indication of a number of repetitions of physical movements that meet the set of one or more movement criteria.

23. A non-transitory computer-readable storage medium storing one or more programs configured to be executed by one or more processors of a computer system that is in communication with a camera and a display generation component, the one or more programs including instructions for:
capturing, via the camera, image data of a physical environment that includes a user of the computer system performing physical movements;
assessing, based on the image data, whether the physical movements meet a set of one or more movement criteria; and
after assessing whether the physical movements meet a set of one or more movement criteria, displaying, via the display generation component, visual feedback based on the image data.

24. A method comprising:
at a computer system that is in communication with a camera and a display generation component:
capturing, via the camera, image data of a physical environment that includes a user of the computer system performing physical movements;
assessing, based on the image data, whether the physical movements meet a set of one or more movement criteria; and
after assessing whether the physical movements meet a set of one or more movement criteria, displaying, via the display generation component, visual feedback based on the image data.
